(19) United States
(12) Patent Application Publication
(10) Pub. No.: US 2026/0364245 A1
(43) Pub. Date: Nov. 19, 2026

(54) ADAPTOR FOR A NEGATIVE PRESSURE WOUND DRESSING

(71) Applicant: ConvaTec Limited, Flintshire (GB)

(72) Inventor: Manjunath Penagondla, Warrington (GB)

(21) Appl. No.: 19/319,542

(22) Filed: Sep. 4, 2025

Related U.S. Application Data
(63) Continuation of application No. PCT/GB2024/050591, filed on Mar. 6, 2024.
(60) Provisional application No. 63/450,476, filed on Mar. 7, 2023.
(60) Provisional application No. 63/514,642, filed on Jul. 20, 2023.
(60) Provisional application No. 63/518,347, filed on Aug. 9, 2023.

(30) Foreign Application Priority Data
Mar. 22, 2023 (GB) 2304150.2
Sep. 12, 2023 (GB) 2313861.3
Sep. 12, 2023 (GB) 2313869.6

(51) Int. Cl. A61M 1/00

(52) U.S. Cl. CPC A61M 1/912

(57) ABSTRACT

An adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient.

### TECHNICAL FIELD OF THE INVENTION

The present invention relates to an adaptor for adapting a surgical wound dressing for use in negative pressure wound therapy.

### BACKGROUND TO THE INVENTION

Wound dressings are known and are generally suitable for treating a variety of wounds, including chronic and acute wound types, such as infected wounds, venous ulcers, diabetic ulcers, burns and surgical wounds.

Negative pressure has been used to treat a range of chronic and acute wounds. Negative pressure may facilitate wound healing through a number of mechanisms, including removal of excess exudate, reduction in periwound edema and increased perfusion. Combined with the physical forces exerted by th negative pressure which draw the wound edges together, this can result in improved wound outcomes. Existing wound dressing systems typically rely on an airway to draw air away from the wound site via a pump.

Many different types of wound dressings are known for aiding in negative pressure wound dressing systems. These different types of wound dressings include many different types of materials and layers, for example, gauze, pads, foam pads or multi-layer wound dressings. However, in each case, the dressing requires a suction port, typically in a backing layer of the dressing, to provide connection to an airway to transmit negative pressure from a pump to the wound site. The suction port is typically formed integral with the wound dressing. The location of the port typically determines the location and orientation of the airway when the airway is attached to the port. Often, this means that the airway is orientated in a position which is disadvantageous for the patient, for example in a position which means that the airway is prone to being pressed against the skin of the patient or in a position where the patient is prone to obstructing the airway by resting upon the airway.

Wound dressings which do not comprise a suction port to provide connection to an airway typically comprise the same general components as that of a negative pressure wound dressing, i.e., a backing layer, an adhesive skin contact layer for adhering the dressing to skin around the wound, and an absorbent structure located between the backing layer and the adhesive skin contact layer. While such dressings function to protect a wound site and absorb a quantity of wound exudate, the wound dressings do not provide the above-mentioned advantages associated with negative pressure wound systems.

Further, the stiffness of the suction port in such close proximity to the wound site can adversely affect the healing process. Patient movement or pressure exerted onto the wound dressing may bring the healing wound into contact with the inflexible suction port of the dressing. Such force can cause disturbance of a wound bed which can damage a wound site. This can potentially cause delays in healing of the wound site and discomfort for the patient.

It is an object of embodiments of the present invention to at least partially overcome or alleviate the above problems and/or to provide means to adapt a wound dressing such that it is capable of transmitting a negative pressure to a wound site without the need for a suction port.

### SUMMARY OF THE INVENTION

The present invention concerns an adaptor for adapting a surgical wound dressing for use in negative pressure wound therapy. The adaptor may comprise an elongate flexible airway. The elongate flexible airway may have an opening at a first end. The airway may comprise a connector at an opposing second end for connection to medical tubing. The airway may comprise an interior lumen between the opening at the first end and the connector at the second end. The airway may comprise a sealing portion having an upper sealing surface and a lower sealing surface. The lower sealing surface of the sealing portion may comprise an adhesive layer and a removable release liner on the adhesive layer.

According to a broad aspect of the present invention, there is provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer.

Advantageously, the present invention provides an adaptor for adapting a surgical wound dressing such that the wound dressing can be used in negative pressure wound therapy. As such, a surgical wound dressing, which may not comprise a suction port, can be adapted to transmit a negative pressure to a wound site to facilitate wound healing.

In addition, the present invention is a simple and cost-effective means for adapting a surgical wound dressing for use in negative pressure wound therapy. In use, the present invention may be arranged between a wound dressing and a patient, such that the upper sealing surface of the sealing portion is adhered to an adhesive skin contact layer of the wound dressing, the lower sealing surface is adhered to the skin of a patient, and the opening at the first end of the airway is arranged at the wound site, within the confides of the adhesive skin contact layer. Beneficially, the upper sealing surface allows for reliable adhesion of the adhesive skin contact layer to the adaptor while still allowing for sufficient adhesion of the wound dressing to the peri wound skin of a patient. Moreover, the adhesive layer comprised on the lower sealing surface allows for reliable adhesion of the adaptor to the peri wound skin of a patient. In use, the medical tubing, to which the connector is connected, may in turn be connected to a source of negative pressure, such as a pump. Activation of the source of negative pressure allows for the passage of air from the wound site, through the opening at the first end of the airway, along the interior lumen toward the second end of the airway, along the connector and the medical tubing, and to the source of negative pressure. Beneficially, as the lower sealing surface comprises an adhesive layer, a complete peripheral seal between the wound dressing and the patient may be formed, therefore, facilitating the creation and maintenance of negative pressure at the wound site.

Advantageously, the present invention allows for the formation of a wound dressing capable of transmitting negative pressure without requiring a suction port. As such, the wound dressing does not suffer from the above disadvantages associated with negative pressure wound dressings comprising a relatively stiff and inflexible suction port in proximity to a wound site. Thus, the present invention can adapt a surgical wound dressing for use in negative pressure wound therapy, the surgical wound dressing being of greater comfort for a patient compared to known negative pressure wound dressings comprising a port.

The upper sealing surface of the sealing portion may be configured, in use, to be adhered to a peripheral adhesive skin contact layer on the underside of the surgical wound dressing. The lower sealing surface may be configured in use to be adhered to the skin of a patient to complete a peripheral seal between the surgical wound dressing and the patient.

Accordingly, in a first aspect of the invention, there is provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient.

Advantageously, the complete peripheral seal between the surgical wound dressing and the patient facilitates the creation of a negative pressure at the wound site and helps to maintain the negative pressure at the wound site for a longer period, therefore facilitating wound healing.

As such, by way of an advantage of the present invention, the complete peripheral seal between the surgical wound dressing and the patient may provide a tight seal around the wound site and a portion of the first end of the airway which comprises the openings. The seal is preferably air-tight, or at least substantially air-tight, to allow for the efficient supply of negative pressure to the wound dressing to aid the wound-healing process. It will be appreciated by those skilled in the art that a completely air-tight seal is ideal, but with a sufficiently powerful pump, the seal need not be absolutely air-tight. As such, in use, the second end of the adaptor may be connected, via a connector and medical tubing as herein described, to a pump. Activation of the pump can draw air from the wound and surrounding wound site within the confines of the peripheral seal, through the openings, along the interior lumen toward the second end of the airway and to the pump where the air may be expelled into the surrounding atmosphere.

Thus, the invention provides for adaptation of a non-negative pressure surgical wound dressing to a surgical wound dressing which can be used in negative pressure wound therapy which, advantageously, facilitates wound healing through a number of mechanisms, including removal of excess exudate, reduction in peri wound edema and increased perfusion. Combined with the physical forces exerted by the negative pressure which draw the wound edges together, this can result in improved wound outcomes.

Moreover, the adaptor of the invention allows for a negative pressure wound therapy system to be provided without using a negative pressure wound dressing. As such, the surgical wound dressings which can be adapted, using the adaptor of the invention, to provide negative pressure wound therapy do not necessarily comprise a port, or similar, in the backing layer of the wound dressing. It follows that the invention provides for a negative pressure wound therapy system without a wound dressing comprising a port. As such, the airway of the adaptor is not restricted in its orientation and the adaptor can be adhered to the surgical wound dressing in a position which is favourable for the patient, for example in a position where the adaptor, and therefore the airway, is not prone to being pressed against the skin of the patient or in a position where the patient is prone to obstructing the airway by resting upon the airway. Further, due to its absence, the relatively stiff suction port is not brought into close proximity with the wound site and therefore does not adversely affect the wound healing process, which could otherwise occur by, for example, the suction port being pressed against the wound which can cause a disturbance to the wound bed and damage the wound site, which may cause delays in healing of the wound site and discomfort for the patient.

The upper sealing surface may be planar or at least substantially planar. Advantageously, this strengthens the adhesion between the adhesive skin contact layer and the sealing portion, therefore, preventing, or at least reducing the probability of, accidental separation of the adaptor from the wound dressing when in use, for example during movement of the patient.

The lower sealing surface may be planar or at least substantially planar. Advantageously, this strengthens the adhesion between the sealing portion and the patient, therefore, preventing, or at least reducing the probability of, accidental separation of the adaptor from the patient when in use, for example during movement of the patient.

Advantageously, in embodiments comprising an upper sealing surface which is planar or at least substantially planar and/or a lower sealing surface which is planar or at least substantially planar, the thickness of the adaptor is reduced. As such, the adaptor is not uncomfortable for a patient, especially when the airway is lent upon and rubs against the patient’s skin. This reduces the risk of the patient developing pressure ulcers and other complications which can result from a wound dressing system being pressed against the skin of a patient.

The maximum width of the airway may be at least two times, three times, four times, five times, six times, seven times, eight times, nine times or ten times the maximum depth of the airway. Beneficially, this greatens the surface area to depth ratio of the airway, therefore, allowing for maximum adhesion of the airway to the patient and/or the adhesive skin contact layer, and minimum discomfort for the patient.

In some embodiments, the airway is formed of a material selected from the group comprising polyurethane, thermoplastic polyurethane, thermoplastic elastomer, polyethylene, polypropylene, polyvinyl chloride, ethylene-vinyl acetate and a silicone material.

Advantageously, each of these materials are cheap and easily obtainable. Further advantageously, each of these materials provide for an airway which is able to be stretched, is flexible and which is relatively soft. As such, the airway does not irritate a patient if the patient’s movement causes the airway to repeatedly rub on their skin. Moreover, these materials, in particular polyurethane, are reusable and recyclable.

The airway may comprise a transmission layer, a top layer and a bottom layer, wherein the top layer is provided over the transmission layer and the bottom layer is provided below the transmission layer.

The top layer and the bottom layer may enclose at least a portion of the transmission layer.

The top layer and the bottom layer may each be constructed from a liquid impermeable material.

The transmission layer may be a foam, a 3D spacer fabric, a knitted material, for example a knitted mesh, for example a nylon knitted mesh, a 3D knitted material, a porous material or a non-woven material. The spacer fabric may be a polyester spacer fabric. The spacer fabric may comprise a microfilament yarn between two fabric layers. The yarn and/or one or each of the fabric layers may be formed of polyester. The foam may be a polyurethane foam.

The transmission layer may be a porous material.

The transmission layer may be a non-woven material.

Advantageously, each of these materials are cheap and readily available. Moreover, many of these materials are recyclable and reusable, and can be formed from recycled materials.

In some embodiments, the transmission layer is formed of a material comprising a plurality of monofilaments separating the upper and lower layers of the transmission layer. In particular, the transmission layer may be formed of a 3D spacer fabric comprising a plurality of monofilaments separating the upper and lower layers.

The 3D spacer fabric may be formed of polyester. The 3D spacer fabric may comprise filaments and yarn each formed of polyester.

The transmission layer may be planar or at least substantially planar.

The transmission layer may be formed by warp knitting, weft knitting or circular knitting.

Advantageously, a transmission layer as described herein, in particular a transmission layer formed of a 3D spacer fabric as described herein, provides superior airflow, greater compression recovery and superior breathability compared to known transmission layers, in particular transmission layers which are not formed of the described 3D spacer fabric. Thus, the airway of the present invention comprising a transmission layer, in particular a transmission layer formed of a 3D spacer fabric, provides an airway which can be of reduced thickness compared to airways of the prior art while also providing superior airflow, compression recovery and breathability. As such, the present invention provides an airway which can be more comfortable for a patient should the airway be rested or lent upon by the patient, and less likely to cause pressure ulcers and other complications which could otherwise result in significant distress and pain for the patient, while providing superior airflow, compression and breathability compared to airways of the prior art.

The airway may comprise a strip of material having a first surface comprising at least one groove, the airway further comprising a film arranged over the first surface of the strip of material and adhered to the strip of material around its longitudinal edges, wherein the at least one groove is provided with at least one projection to form at least two channels between the strip and the film.

The at least one groove may be provided with a plurality of projections, such that there is provided a plurality of channels between the strip and the film.

A plurality of channels has the advantage of increasing the number of routes available to fluid flowing along the airway. This means that, in the event the airway is bent, twisted or kinked, it is probable that flow of fluid along the airway would not be completely obstructed as at least some channels would remain at least partially open to the flow of fluid. Thus, the flow of fluid along the airway of the invention is maintained and is more reliable even if the airway is bent, twisted or kinked, compared to airways of the prior art.

The plurality of projections may comprise any number of projections, for example about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or about 250 projections.

The plurality of projections may comprise at least about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or at least about 250 projections.

The plurality of projections may comprise no more than about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, or no more than about 250 projections.

The plurality of channels may form a network of interconnected channels.

The plurality of channels may be arranged as an interlaced pattern of channels. By ‘interlaced’, we mean that each channel crosses at least one other channel. Beneficially, an interlaced pattern of channels is less likely to be blocked by bending/folding compared to, for example, a spacer material or a plurality of channels arranged in parallel to one another.

The interlaced pattern of channels may comprise a plurality of criss-crossed channels. By this we mean that there is significant crossing of each channel with at least one other channel. Advantageously, an interlaced pattern of channels, for example comprising a plurality of criss-crossed channels, increases the overall channel volume of the airway. Beneficially, this means that a greater volume of fluid (for example air) can be transmitted along the airway and, therefore, the adaptor, at any given time compared to airways of the prior art which do not comprise a plurality of channels, in particular an interlaced pattern of channels. Further advantageously, an interlaced pattern of channels maintains a favourable flow rate of fluid along the airway during compression or load upon the airway. As such, for example when used for transmitting a negative pressure to a wound site, the airway of the invention maintains a negative pressure at a wound site even when the airway has been folded and/or compressed by a user.

The plurality of channels may be arranged as a zig-zag pattern of channels. By this we mean that each channel is arranged in a zig-zag orientation and that each channel does not cross another channel.

At least two of the channels may be in fluid communication with one another.

Advantageously, this means that the overall channel volume of the airway and the number of routes that fluid can take when flowing along the airway is increased which in turn means that the flow of fluid to and/or from a wound site is increased. In some embodiments, all of the channels are in fluid communication with one another, which further enhances the aforementioned advantage.

The at least two channels may be in fluid communication with the opening.

The at least one projections may be discrete, individual projections. The discrete, individual projections may each have a cross-section (when viewed in plan view) which is any shape but in particular may be circle, oval, triangle, square, diamond, rhombus, parallelogram, rectangle, pentagon, hexagon, heptagon or octagon.

The or each projection may be an elongated projection which extends at least part way along the length of the strip of material. The or each elongated projection may be a linear projection or may be in a zig-zag. The plurality of projections may comprise at least one discrete, separate projection and at least one elongated projection extending at least part way along the length of the strip of material.

The or each elongate projection may extend at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or at least about 95% along the length of the strip of material.

The or each projection may extend from a base of the or each groove.

The or each projection may extend to a height equal to, or at least substantially equal to, the height of the strip of material.

The or each projection may abut the film when the film is adhered to the first surface of the strip of material. The film may be adhered to one or more of the at least one projections. For example, the or each projection may comprise an upper surface which is adhered to a lower surface of the film. The film may be adhered to one or more of the at least one projections by heat lamination. In known airways, despite comprising a spacer material, when in use, the films used to sandwich the spacer material often collapse inwardly toward each other causing an obstruction to the flow of fluid through the airway. Advantageously, in respect of the present invention, in use, the least one projection prevent the film collapsing into the at least one or plurality of grooves and therefore obstructing the flow of fluid through the airway.

The film may be substantially planar. Advantageously, this means that the airway has at least one substantially planar surface which makes the airway more comfortable should a user sit or rest upon the airway.

The strip of material may have a width of about 5 mm, about 10 mm, about 15 mm, about 20 mm, about 25 mm, about 30 mm, about 35 mm, about 40 mm, about 45 mm, or about 50 mm.

The strip of material may have a width of at least about 5 mm, about 10 mm, about 15 mm, about 20 mm, about 25 mm, about 30 mm, about 35 mm, about 40 mm, about 45 mm, or at least about 50 mm.

The strip of material may have a width of no more than about 5 mm, about 10 mm, about 15 mm, about 20 mm, about 25 mm, about 30 mm, about 35 mm, about 40 mm, about 45 mm, or no more than about 50 mm.

The strip of material may have a depth of about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or about 3.0 mm.

The strip of material may have a depth of at least about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or at least about 3.0 mm.

The strip of material may have a depth of no more than about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or no more than about 3.0 mm.

The depth of the strip of material is measured from the base of the material to its highest point, e.g., to the top of a projection.

The or each groove may have a depth of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, or about 2.9 mm.

The or each groove may have a depth of at least about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, or at least about 2.9 mm.

The or each groove may have a depth of no more than about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, or no more than about 2.9 mm.

The or each groove may have a depth of between about 0.1 mm and about 2.9 mm, about 0.2 mm and about 2.8 mm, about 0.3 mm and about 2.6 mm, about 0.4 mm and about 2.4 mm, about 0.5 mm and about 2.2 mm, about 0.5 mm and about 2.0 mm, about 0.6 mm and about 1.8 mm, about 0.7 mm and about 1.6 mm, about 0.8 mm and about 1.4 mm, about 0.9 mm and about 1.2 mm, or about 1.0 mm.

Advantageously, the or each groove has a depth which is significantly less than the depth of a spacer material commonly found in known airways. As such, the present invention provides for an airway which has reduced depth compared to that of airways of the prior art. Thus, the airway of the present invention is more comfortable for a patient should they lie upon the airway.

By ‘depth’ of a groove, we mean the distance between the uppermost surface or point of a projection and the base of the groove from which the projection extends.

The or each groove may have a width of about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or about 3.0 mm.

The or each groove may have a width of at least about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or at least about 3.0 mm.

The or each groove may have a width of no more than about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or no more than about 3.0 mm.

The or each groove may have a width of between about 0.1 mm and about 3.0 mm, about 0.2 mm and about 2.8 mm, about 0.3 mm and about 2.6 mm, about 0.4 mm and about 2.4 mm, about 0.5 mm and about 2.2 mm, about 0.5 mm and about 2.0 mm, about 0.6 mm and about 1.8 mm, about 0.7 mm and about 1.6 mm, about 0.8 mm and about 1.4 mm, about 0.9 mm and about 1.2 mm, or about 1.0 mm.

Advantageously, grooves having the above-mentioned depth and/or width dimensions allow for the sufficient flow of fluid (typically air) away from the wound site when used as part of a negative pressure wound dressing. Such dimensions of the grooves are typically smaller than the dimensions of a spacer material used in known airways. The present invention therefore provides an airway which may be less cumbersome for the patient and does not cause the patient discomfort should they lie upon the airway when in use.

Further advantageously, the abovementioned depth and/or width dimensions of the or each groove further reduce the ability of the film to compress into the or each groove if the airway is, for example, folded upon itself or is put under the weight of the user, when in use.

By ‘width’ of a groove, we mean the distance between adjacent projections, between which is a groove.

In embodiments comprising more than one groove, not all of the grooves may have the same width. The average width of the grooves may be about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or about 3.0 mm.

The average width of the grooves may be at least about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or at least about 3.0 mm.

The average width of the grooves may be no more than about 0.1 mm, about 0.2 mm, about 0.3 mm, about 0.4 mm, about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, about 2.2 mm, about 2.3 mm, about 2.4 mm, about 2.5 mm, about 2.6 mm, about 2.7 mm, about 2.8 mm, about 2.9 mm or no more than about 3.0 mm.

The average width of the grooves may be between about 0.1 mm and about 3.0 mm, about 0.2 mm and about 2.8 mm, about 0.3 mm and about 2.6 mm, about 0.4 mm and about 2.4 mm, about 0.5 mm and about 2.2 mm, about 0.5 mm and about 2.0 mm, about 0.6 mm and about 1.8 mm, about 0.7 mm and about 1.6 mm, about 0.8 mm and about 1.4 mm, about 0.9 mm and about 1.2 mm, or about 1.0 mm.

The film may be formed of a material selected from the group comprising polyurethane, thermoplastic polyurethane, thermoplastic elastomer, polyethylene, polypropylene, polyvinyl chloride, ethylene-vinyl acetate and a silicone material, for example a silicone rubber.

The strip may be formed of a material selected from the group comprising polyurethane, thermoplastic polyurethane, thermoplastic elastomer, polyethylene, polypropylene, polyvinyl chloride, ethylene-vinyl acetate and a silicone material, for example a silicone rubber.

Advantageously, each of these materials are cheap and easily obtainable. Further advantageously, each of these materials provide for an airway which is able to be stretched, is flexible and which is relatively soft. As such, the airway does not irritate a patient if the patient’s movement causes the airway to repeatedly rub on their skin. Moreover, these materials, in particular polyurethane, are reusable and recyclable.

The airway may have a generally planer exterior surface.

The strip of material and/or the film may be formed of a transparent or translucent material. Advantageously, this means that the airway is transparent or translucent, respectively, so that if a blockage occurs within the airway, for example clogging due to wound exudate, then this is visible by the patient or a healthcare professional.

The airway may comprise a filter, an infection detection indicator and/or a dressing change indicator. The filter, an infection detection indicator and/or a dressing change indicator may be arranged toward the first end of the airway, such as proximal to the opening at the first end.

The filter may be impermeable to liquids but permeable to gases. Advantageously, the filter may function as a liquid barrier to ensure that no wound exudate can exit the wound site and pass along the airway.

The filter may function as a bacterial barrier.

The filter may be a microporous membrane. The microporous membrane may be any polymeric material. The filter may be a microporous hydrophobic membrane (MHM). The MHM may be formed from one or more of PTFE, polypropylene, PVDF and acrylic copolymer. Each of these optional polymers can be treated in order to obtain specific surface characteristics that can be both hydrophobic and oleophobic. As such, these will repel liquids with low surface tensions such as multi-vitamin infusions, lipids, surfactants, oils and organic solvents.

The filter may comprise an odour absorbent material, for example activated charcoal or carbon fibre cloth.

The filter may have a pore size of about 0.1μm, about 0.2μm, about 0.3μm, about 0.4μm, about 0.5μm, about 0.6μm, about 0.7μm, about 0.8μm, about 0.9μm or about 1.0μm.

The filter may comprise an oleophobic filter membrane. Beneficially, the oleophobic filter membrane may prevent any lipids found in wound exudate from blocking the filter.

The infection detection indicator and/or the dressing change indicator may comprise an auxiliary compound. The auxiliary compound may be an antimicrobial, antifungal, anti-inflammatory, or any other such therapeutic compounds. In some embodiments, the indicator may be by means of a colour change. The colour may change as a function of time (e.g., to indicate when the dressing needs to be changed), if the dressing is saturated, or if the dressing has absorbed a certain amount of a harmful substance (e.g., to indicate the presence of infectious agents).

In some embodiments, the at least two channels may comprise at least a first channel and a second channel.

The first channel and the second channel may be distinct from one another such that the first channel is not in fluid communication with the second channel. This arrangement provides an airway which allows for the passage of fluid in two directions, i.e., the passage of fluid away from a wound site and the passage of fluid toward a wound site.

The plurality of channels may comprise at least a first channelled area and a second channelled area, each channelled area comprising at least one channel. The first channelled area may be distinct from the second channelled area such that the first channelled area is not in fluid communication with the second channelled area.

The first channelled area and the second channelled area may be separated by a seal between the strip and the film. This arrangement provides an airway which allows for the passage of fluid in two directions, i.e., the passage of fluid away from a wound site and the passage of fluid toward a wound site. The seal may be an adhesion between the film and one or more projections. The seal may penetrate into one or more grooves. The seal may be formed by lamination of the film to the strip. The seal may be a longitudinal seal extending lengthways along the airway and arranged to separate the first channelled area from the second channelled area so that the two channelled areas are not in fluid communication with one another. In this embodiment, the airway may comprise two openings at the first end, and a first opening may be arranged in fluid communication with the first channelled area, and a second opening may be arranged in fluid communication with the second channelled area. Advantageously, this means that the airway can provide the passage of fluid in two different directions, i.e., a first direction, for example toward the wound site, via the first channelled area, and a second direction, for example away from the wound site, via the second channelled area.

The airway may comprise a second elongated strip of material. The second elongated strip of material may comprise any of the features, optional or otherwise, associated with the (first) elongated strip of material as described above.

The second elongated strip of material may comprise a first end. The second elongated strip of material may comprise a second end. The second elongated strip of material may comprise a pair of longitudinal edges, i.e., a first longitudinal edge and a second longitudinal edge. The longitudinal edges are joined at the first end by an outwardly curved portion, the outermost part of which may form the terminus of the first end. The longitudinal edges may be joined by an outwardly curved portion, the outermost part of which may form the terminus of the second end. The second end may be distal to the first end.

The second strip of material may have a width (i.e., the distance between the first longitudinal edge and the second longitudinal edge) of about 15 mm and a length (i.e., the distance from the terminus of the first end to the terminus of the second end) of about 300 mm.

The airway may comprise a third opening. The third opening may be arranged at the first end of the second strip of material. The third opening may be in the shape of a cross. The third opening may be arranged central relative to the periphery of the first end of the second strip of material. The third opening may extend through the entire of the depth of the second strip of material.

The airway may comprise a fourth opening. The fourth opening may be arranged at the second end of the second strip of material. The fourth opening may be in the shape of a cross. The fourth opening may be arranged central relative to the periphery of the second end of the second strip of material. The fourth opening may extend through the entire of the depth of the second strip of material.

The second strip of material may comprise a downwardly facing first surface and an upwardly facing second surface, when in use. In use, the first surface may face toward the wound site and the second surface may face away from the wound site.

The first surface of the second strip of material may comprise a grooved area comprising at least one groove, preferably a plurality of grooves. The grooved area may comprise at least one projection, preferably a plurality of projections.

The grooved area of the first surface of the second strip of material may comprise each of the features of the grooved area of the first surface of the first strip of material.

The grooved area may have a width which is equal to about 60% of the width of the second strip of material, and a length which is equal to about 90% of the length of the second strip of material. The grooved area may be substantially rectangular.

The at least one groove may be arranged as an interlaced pattern of criss-crossed grooves, with each groove being separated from another groove, or another portion of a groove, by a projection. The or each projection may extend from the base of a groove. The or each groove may have a width of 1.0 mm and a depth of 1.5 mm. The or each groove may be formed by laser engraving/etching of the strip of material. The strip of material may have a depth of 2.0 mm.

In embodiments comprising a plurality of grooves, each groove may be in fluid communication with each other groove such that the plurality of grooves forms an interconnected lattice of grooves in fluid communication with one another. Advantageously, in this embodiment, the plurality of grooves increases the overall volume of the grooves of the airway. In turn, this means that a greater volume of fluid (for example air) is transmitted along the airway at any given time compared to airways of the prior art which do not comprise a plurality of grooves, in particular an interlaced pattern of grooves.

Further, when in use, the interconnected lattice of grooves maintains a favourable flow rate of fluid along the airway during compression or load upon the airway. As such, for example when used for transmitting a negative pressure to a wound site, the airway of the invention maintains a negative pressure at a wound site even when the airway has been folded and/or compressed by a user.

The second end of the second strip of material may comprise an opening in the shape of a cross. The opening may be arranged central relative to the periphery of the second end.

At least one groove may be in fluid communication with the opening at the first end of the second strip of material, and with the opening at the second end of the second strip of material.

The airway may comprise the second strip of material such that first surface of the second strip of material is arranged over the film. The first surface of the second strip of material may be adhered to the film (for example by an adhesive, alternatively heat lamination may be used) about its longitudinal edges. Once the second strip of material is adhered to the film, the plurality of grooves may form at least one second channel, preferably a second plurality of channels, between the second strip of material and the film. In embodiments comprising a second plurality of channels, the second plurality of channels may be in fluid communication with each other channel such that the second plurality of channels forms a network of interconnected channels in fluid communication with one another. The interconnected channels may be in fluid communication with the opening at the first end of the second strip of material and with the opening at the second end of the second strip of material.

Advantageously, this arrangement allows for at least two channels (i.e., at least a first channel and at least a second channel) to be formed which are not in fluid communication with one another. This arrangement provides an airway which allows for the passage of fluid in two directions, i.e., the passage of fluid away from a wound site and the passage of fluid toward a wound site.

In some embodiments, the channel(s) between the first strip of material and the film are not in fluid communication with the channel(s) between the second strip of material and the film.

Advantageously, the embodiment provides an airway which allows for the passage of fluid, for example air, in two directions simultaneously, i.e., the passage of fluid away from a wound site and the passage of fluid toward a wound site. Thus, this may facilitate wound healing through a number of mechanisms, including removal of excess exudate, reduction in periwound edema and increased perfusion, therefore, resulting in improved wound outcomes.

The connector may be a first connector for connection of the channel(s) between the first strip of material and the film to a first medical tubing and an interior lumen therebetween.

The airway may comprise a second connector for connection of the channel(s) between the second strip of material and the film to a second medical tubing and an interior lumen therebetween.

The first and/or second connector may be a Luer or barb connector. The first and/or second connector may be connectable to a pump.

The first strip of material may comprise the lower sealing surface. The second strip of material may comprise the upper sealing surface.

The opening may be a first opening and the channel(s) between the first strip of material and the film may be in fluid communication with the first opening.

The airway may comprise a second opening. The second opening may be arranged at the second end of the first strip of material. The second opening may be in the shape of a cross. The second opening may be arranged central relative to the periphery of the second end of the first strip of material. The second opening may extend through the entire of the depth of the first strip of material.

The channel(s) between the first strip of material and the film may be in fluid communication with the second opening. Thus, the second opening may be in fluid communication with the first opening, via the or each first channel.

The channel(s) between the second strip of material and the film may be in fluid communication with the third opening and/or the fourth opening. Thus, the third opening may be in fluid communication with the fourth opening, via the or each second channel.

In use, the first opening may be arranged proximal to, for example arranged facing, the wound site.

In use, the third opening may be arranged proximal to, for example arranged facing, the wound dressing, for example, proximal to, for example arranged facing, a wound contact layer of the wound dressing.

The first opening may be separated from the third opening by the film. The first opening may be separated from the third opening by the film such that the first opening is not in fluid communication with the third opening.

The second opening may be separated from the fourth opening by the film. The second opening may be separated from the fourth opening by the film such that the second opening is not in fluid communication with the fourth opening.

The film between the first strip of material and the second strip of material may comprise a plurality of layers of film bonded together, for example by heat lamination or an adhesive.

In some embodiments, the film is formed of a material selected from the group comprising polyurethane, thermoplastic polyurethane, thermoplastic elastomer, polyethylene, polypropylene, polyvinyl chloride, ethylene-vinyl acetate and a silicone material.

The second strip of material may be formed of the same material as the first strip of material.

The length of the airway from the terminus of the first end to the sealing portion may be about 1 cm, about 2 cm, about 3 cm, about 4 cm, about 5 cm, about 6 cm, about 7 cm, about 8 cm, about 9 cm, or about 10 cm.

The length of the airway from the terminus of the first end to the sealing portion may be at least about 1 cm, about 2 cm, about 3 cm, about 4 cm, about 5 cm, about 6 cm, about 7 cm, about 8 cm, about 9 cm, or at least about 10 cm.

The length of the airway from the terminus of the first end to the sealing portion may be no more than about 1 cm, about 2 cm, about 3 cm, about 4 cm, about 5 cm, about 6 cm, about 7 cm, about 8 cm, about 9 cm, or no more than about 10 cm.

The or each first opening, second opening, third opening and/or fourth opening may comprise a plurality of openings, each opening being in communication with the interior lumen.

The first end of the first and/or second strip of material may comprise from 2 to 18 openings, from 6 to 15 openings, or from 9 to 12 openings.

The first end of the first and/or second strip of material may comprise about 2, about 3, about 4, about 5, about 6, about 7, about 8, about 9, about 10, about 11, about 12, about 13, about 14, about 15, about 16, about 17 or about 18 openings.

The first end of the first and/or second strip of material may comprise at least about 2, about 3, about 4, about 5, about 6, about 7, about 8, about 9, about 10, about 11, about 12, about 13, about 14, about 15, about 16, about 17 or at least about 18 openings.

The first end of the first and/or second strip of material may comprise no more than about 2, about 3, about 4, about 5, about 6, about 7, about 8, about 9, about 10, about 11, about 12, about 13, about 14, about 15, about 16, about 17 or no more than about 18 openings.

The shape of the or each opening may be a circle, square, rectangle, rhombus, triangle, pentagon, hexagon, heptagon, octagon, or in the shape of a cross or in the form of a slit.

The or each opening may have a diameter or a longest dimension of about 0.1cm, about 0.2cm, about 0.3cm, about 0.4cm, about 0.5cm, about 0.6cm, about 0.7cm, about 0.8cm, about 0.9cm, or about 1.0cm.

The or each opening may have a diameter or a longest dimension of at least about 0.1cm, about 0.2cm, about 0.3cm, about 0.4cm, about 0.5cm, about 0.6cm, about 0.7cm, about 0.8cm, about 0.9cm, or at least about 1.0cm.

The or each opening may have a diameter or a longest dimension of no more than about 0.1cm, about 0.2cm, about 0.3cm, about 0.4cm, about 0.5cm, about 0.6cm, about 0.7cm, about 0.8cm, about 0.9cm, or no more than about 1.0cm.

The sealing portion may comprise a first wing and a second wing, wherein each wing extends outwardly from the first end of the airway.

Thus, in some embodiments, there may be provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient, wherein the sealing portion comprises a first wing and a second wing, wherein each wing extends outwardly from the first end of the airway.

The upper sealing surface of each of the first wing and second wing may extend outwardly from an uppermost surface of the airway.

The lower sealing surface of each of the first wing and second wing may extend outwardly from a lowermost surface of the airway.

Thus, in some embodiments, there may be provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient, wherein the sealing portion comprises a first wing and a second wing, wherein each wing extends outwardly from the first end of the airway, further wherein the upper sealing surface of each of the first wing and second wing extends outwardly from an uppermost surface of the airway, and the lower sealing surface of each of the first wing and second wing extends outwardly from a lowermost surface of the airway.

The upper sealing surface of the first wing and lower sealing surface of the first wing may converge toward each other.

Thus, in some embodiments, there may be provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient, wherein the sealing portion comprises a first wing and a second wing, wherein each wing extends outwardly from the first end of the airway, further wherein the upper sealing surface of each of the first wing and second wing extends outwardly from an uppermost surface of the airway, and the lower sealing surface of each of the first wing and second wing extends outwardly from a lowermost surface of the airway, further wherein the upper sealing surface of the first wing and lower sealing surface of the first wing converge toward each other.

The upper sealing surface of the second wing and lower sealing surface of the second wing may converge toward each other.

Thus, in some embodiments, there may be provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient, wherein the sealing portion comprises a first wing and a second wing, wherein each wing extends outwardly from the first end of the airway, further wherein the upper sealing surface of each of the first wing and second wing extends outwardly from an uppermost surface of the airway, and the lower sealing surface of each of the first wing and second wing extends outwardly from a lowermost surface of the airway, further wherein the upper sealing surface of the second wing and lower sealing surface of the second wing converge toward each other.

In some embodiments, there may be provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient, wherein the sealing portion comprises a first wing and a second wing, wherein each wing extends outwardly from the first end of the airway, further wherein the upper sealing surface of each of the first wing and second wing extends outwardly from an uppermost surface of the airway, and the lower sealing surface of each of the first wing and second wing extends outwardly from a lowermost surface of the airway, further wherein the upper sealing surface of the first wing and lower sealing surface of the first wing converge toward each other, further wherein the upper sealing surface of the second wing and lower sealing surface of the second wing converge toward each other.

The lower sealing surface of the first wing and the lower sealing surface of the second wing may each be planar.

Thus, in some embodiments, there may be provided an adaptor for adapting a surgical wound dressing for use in negative pressure wound therapy, the adaptor comprising an elongate flexible airway having an opening at a first end; a connector at an opposing second end for connection to medical tubing and an interior lumen therebetween; the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient, wherein the sealing portion comprises a first wing and a second wing, wherein each wing extends outwardly from the first end of the airway, further wherein the upper sealing surface of each of the first wing and second wing extends outwardly from an uppermost surface of the airway, and the lower sealing surface of each of the first wing and second wing extends outwardly from a lowermost surface of the airway, further wherein the lower sealing surface of the first wing and the lower sealing surface of the second wing are each planar.

Advantageously, the first wing and the second wing as described herein allow for easy insertion between an adhesive skin contact layer and the peri wound skin of a patient. As such, the adaptor of the invention may be used with a surgical wound dressing which is already adhered to a patient. Thus, in use, a portion of the adhesive skin contact layer of the surgical wound dressing can be detached from the patient and the removable release liner on the adhesive layer of the lower sealing surface may be removed to expose the adhesive layer. The first end of the adaptor may then be inserted between the detached portion of the adhesive skin contact layer and the patient such that the opening at the first end is proximal to the wound site and the upper sealing surface of the sealing portion is between the adhesive skin contact layer and the patient. The detached portion of the adhesive skin contact layer may then be adhered to the upper sealing surface of the sealing portion, and the lower sealing surface of the sealing portion can be adhered to the patient. Thus, a complete peripheral seal between the surgical wound dressing and the patient is formed.

The first wing and/or the second wing may be a separate part to the airway or formed integral with the airway. The first wing and/or the second wing may be overmoulded or thermally formed.

Each wing may extend outwardly a distance of about 1 cm, about 2 cm, about 3 cm, about 4 cm, about 5 cm, about 6 cm, about 7 cm, about 8 cm, about 9 cm, or about 10 cm.

Each wing may extend outwardly a distance of no more than about 1 cm, about 2 cm, about 3 cm, about 4 cm, about 5 cm, about 6 cm, about 7 cm, about 8 cm, about 9 cm, or no more than about 10 cm.

Each wing may extend outwardly a distance at least about 1 cm, about 2 cm, about 3 cm, about 4 cm, about 5 cm, about 6 cm, about 7 cm, about 8 cm, about 9 cm, or at least about 10 cm.

The connector may be a Luer or barb connector. The connector may be connectable to a pump.

According to a second aspect of the invention, there is provided a method of adapting a surgical wound dressing for use in negative pressure wound therapy, the surgical wound dressing having at least a peripheral adhesive skin contact layer on the underside of the surgical wound dressing, the method comprising:
a . providing an adaptor according to the first aspect of the invention,
b. removing the release liner from the adhesive layer of the lower sealing surface of the airway; and
c. adhering the adaptor to an underside of a surgical wound dressing, such that the upper sealing surface of the sealing portion is adhered to the peripheral adhesive skin contact layer.

The method may further comprise adhering the peripheral adhesive skin contact layer of the surgical wound dressing to the periwound skin of a patient.

The adhesive layer of the sealing portion and the adhesive periphery of the wound dressing together may form a continuous adhesive boundary around a wound. Advantageously, the continuous adhesive boundary between the surgical wound dressing and the patient facilitates the creation of a negative pressure at the wound site and helps to maintain the negative pressure at the wound site for a longer period, therefore facilitating wound healing.

The invention according to the second aspect may include any of or more of the features, optional or otherwise, of the invention according to the first aspect.

According to a third aspect of the invention, there is provided a use of an adaptor according to the first aspect in adapting a surgical wound dressing to a negative pressure wound dressing for use in negative pressure wound therapy.

The invention according to the third aspect may include any of or more of the features, optional or otherwise, of the invention according to the first or second aspect.

### DETAILED DESCRIPTION OF THE INVENTION

In order that the invention may be more clearly understood one or more embodiments thereof will now be described, by way of example only, with reference to the accompanying drawings, of which:

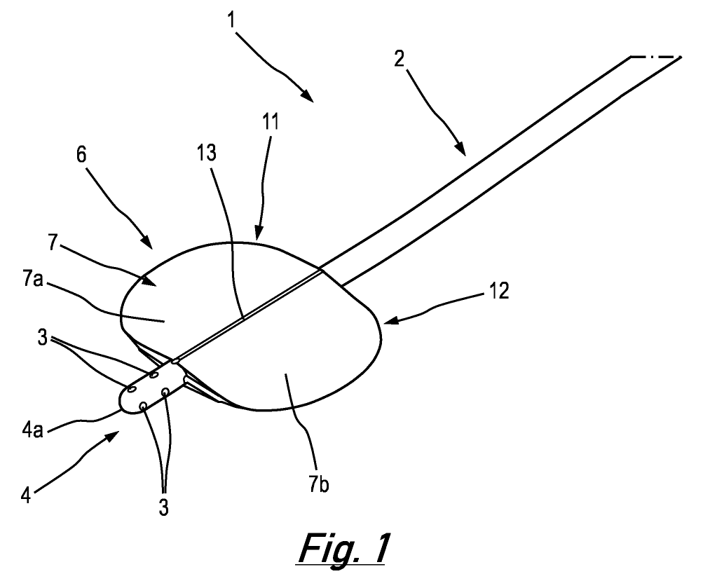

FIG. 1 is a perspective view of a part of an adaptor according to a first embodiment;

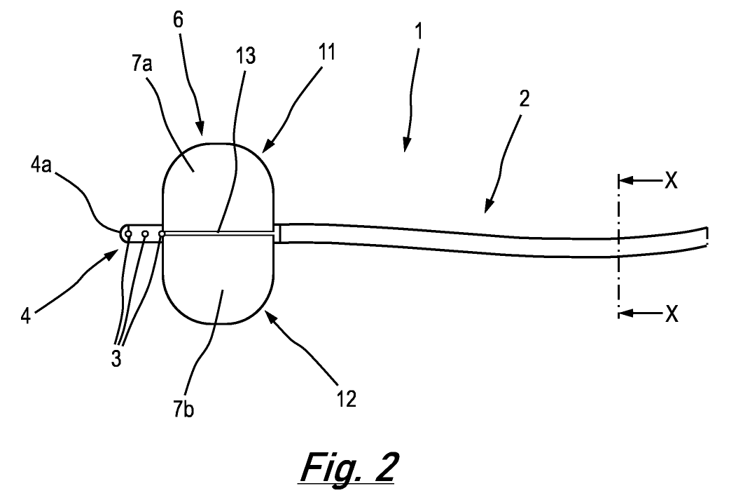

FIG. 2 is a plan view of the adaptor of FIG. 1;

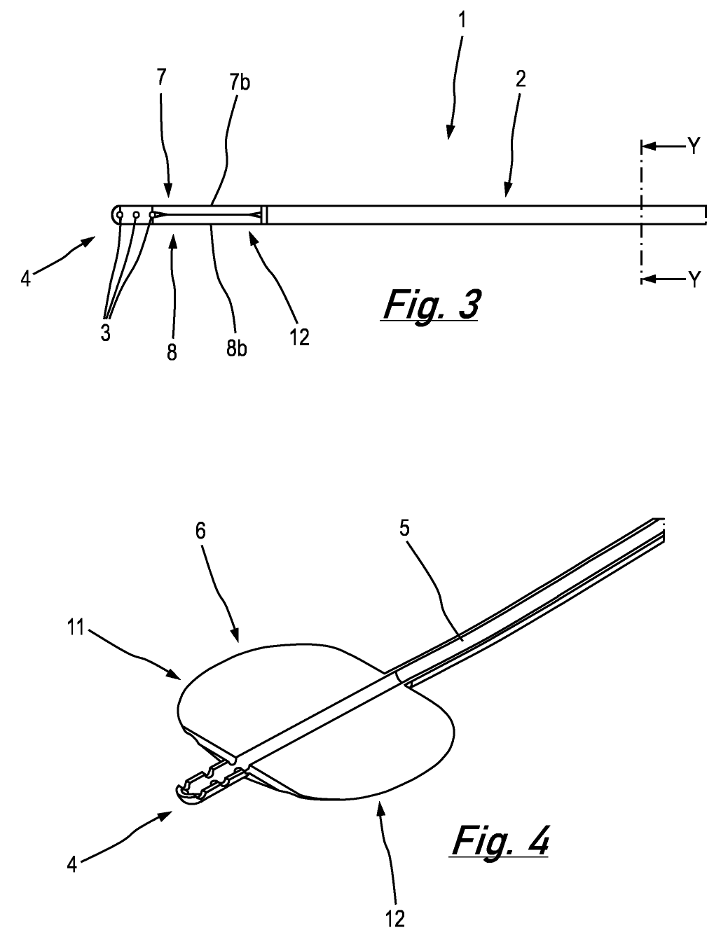

FIG. 3 is a side view of the adaptor of FIG. 1;

FIG. 4 is a perspective view of the adaptor of FIG. 1 with a portion of the adaptor cut away along line X-X shown in FIG. 2;

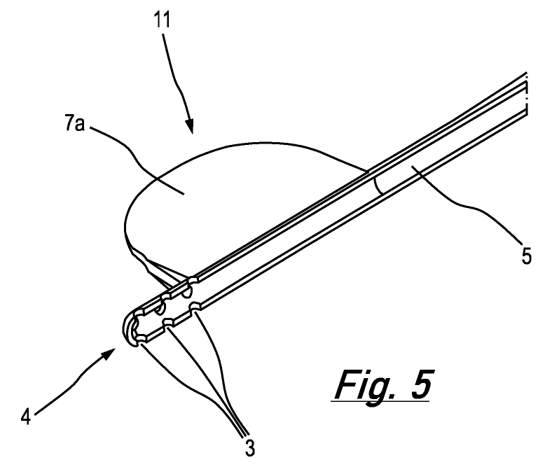

FIG. 5 is a perspective view of the adaptor of FIG. 1 with a portion of the adaptor cut away along line Y-Y shown in FIG. 3;

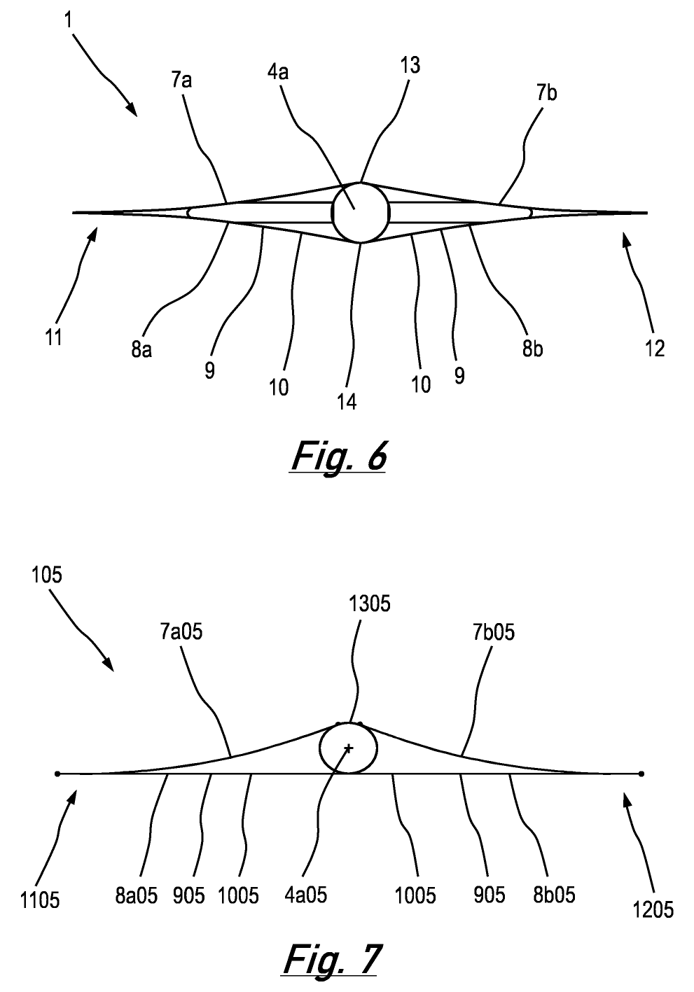

FIG. 6 is a front view of the adaptor of FIG. 1;

FIG. 7 is a front view of a second embodiment of the adaptor of FIG. 1;

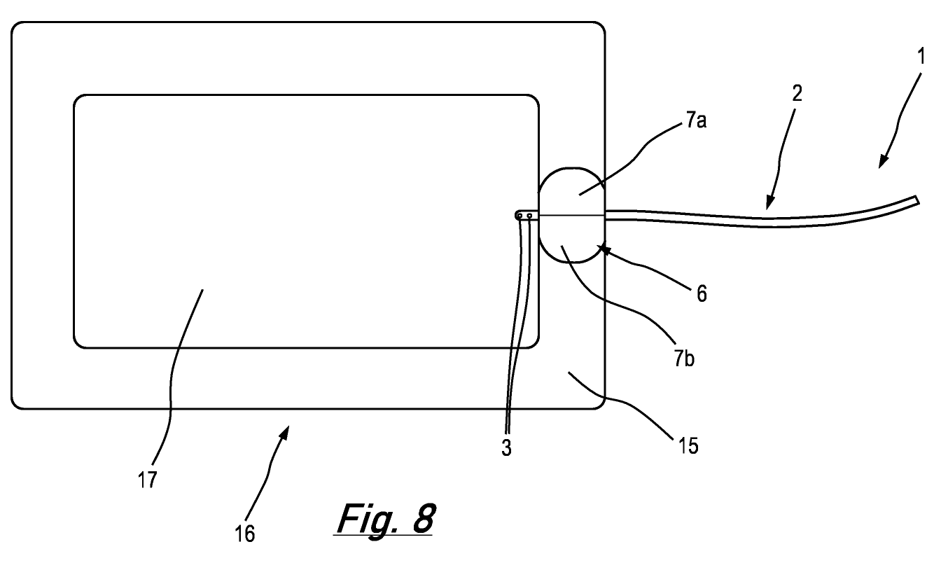

FIG. 8 is a plan view of a surgical wound dressing with the adaptor of FIG. 1 when in use;

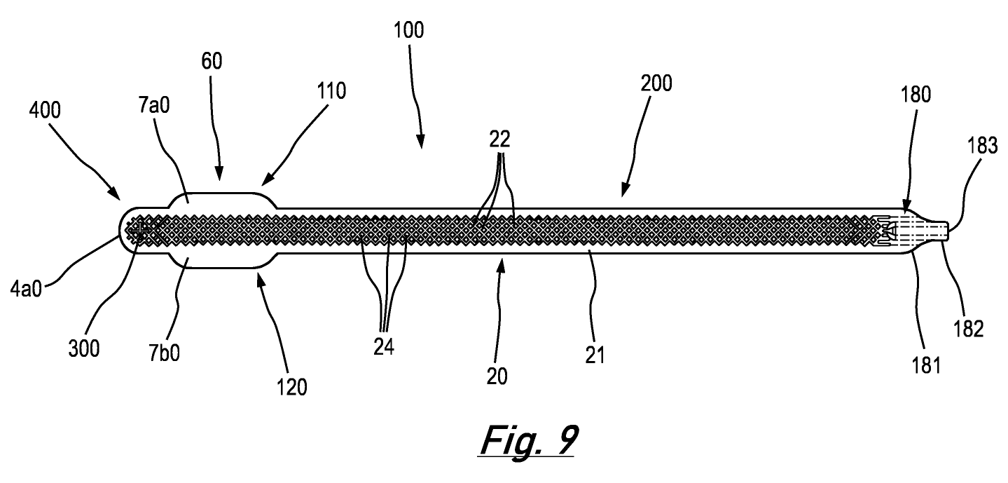

FIG. 9 is a plan view of an adaptor according to a third embodiment;

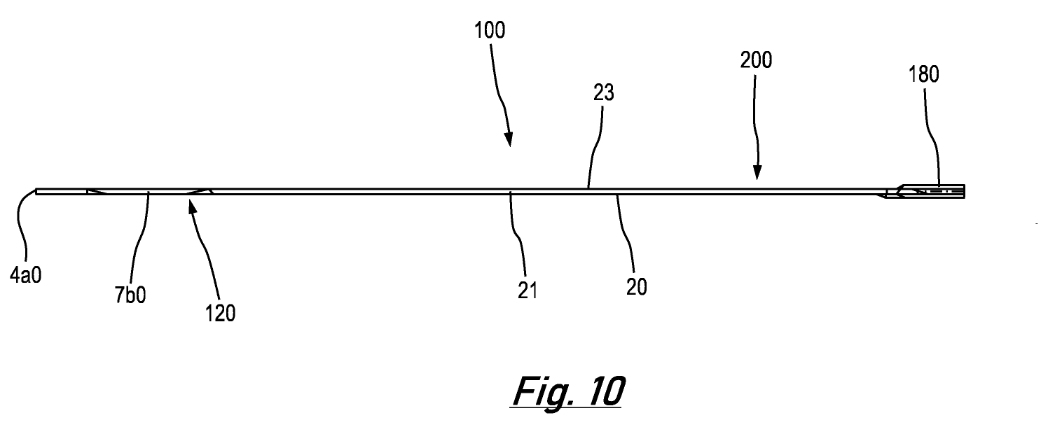

FIG. 10 is a side view of the adaptor of FIG. 9;

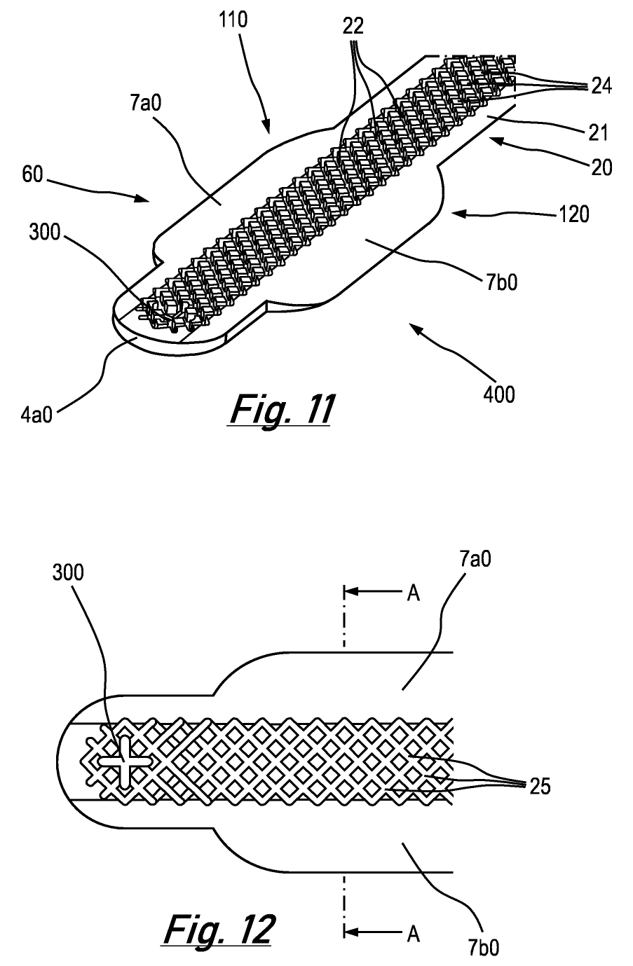

FIG. 11 is a perspective view of part of the adaptor of FIG. 9 with the film not shown;

FIG. 12 is a plan view of the part of the adaptor of FIG. 9;

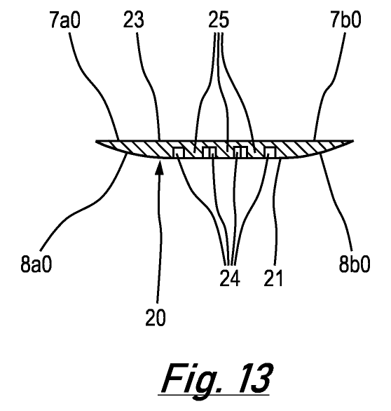

FIG. 13 is a cross section view along line A-A of the part of the adaptor of FIG. 12;

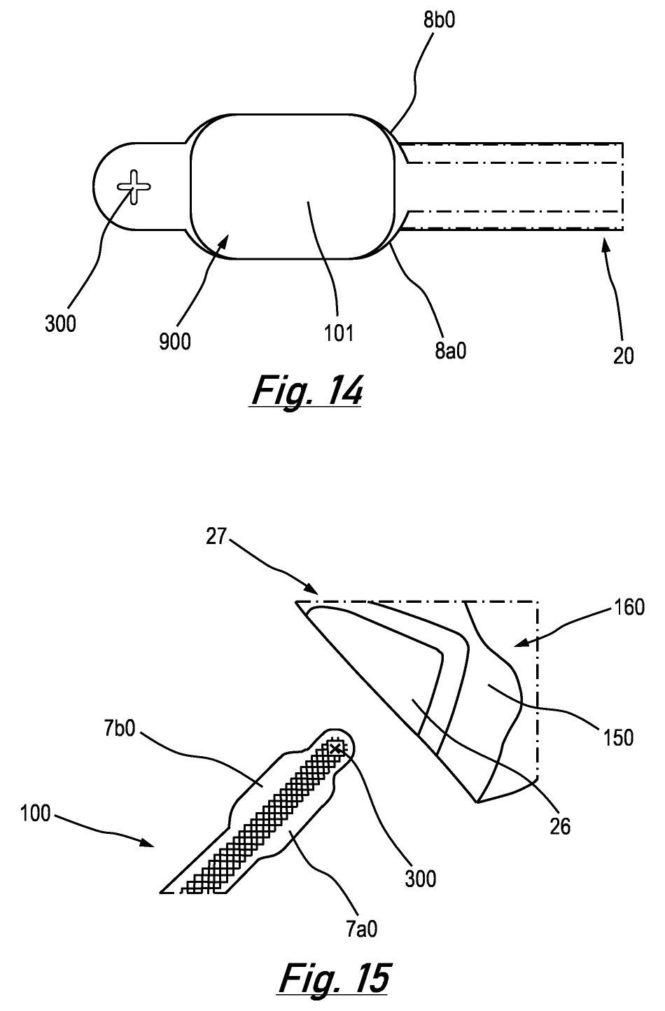

FIG. 14 is an underside view of part of the adaptor of FIG. 9;

FIG. 15 is a photograph showing part of the adaptor of FIG. 9 and a surgical wound dressing;

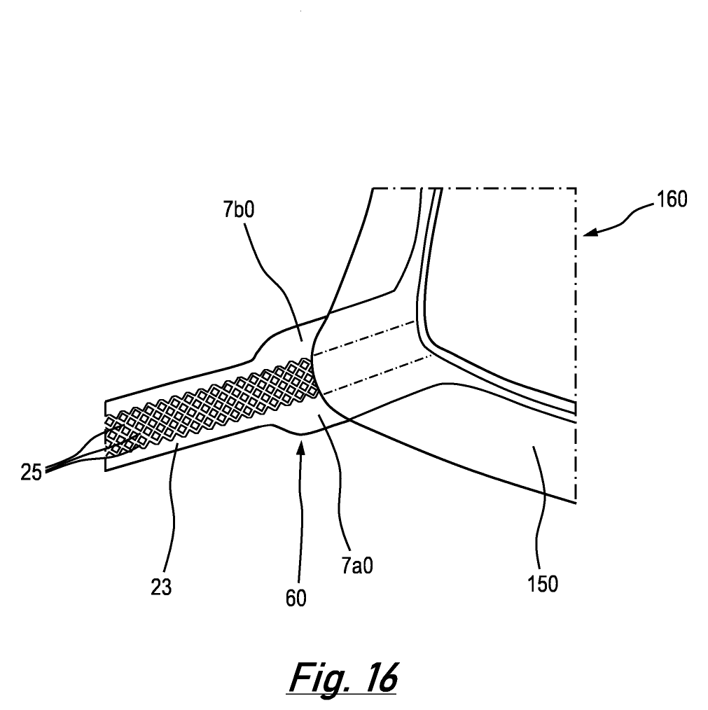

FIG. 16 is a photograph showing part of the adaptor of FIG. 9 when adhered to a surgical wound dressing;

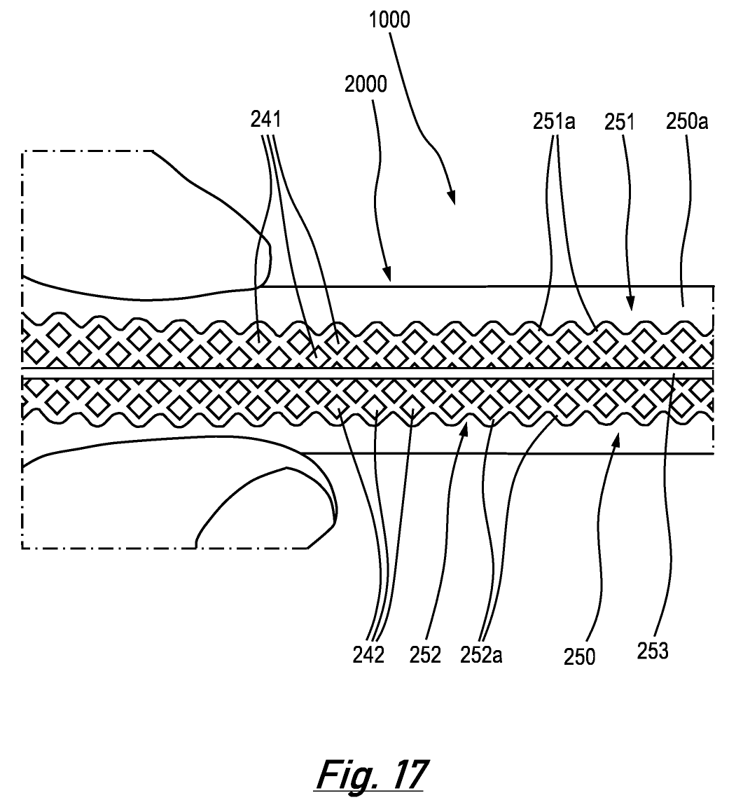

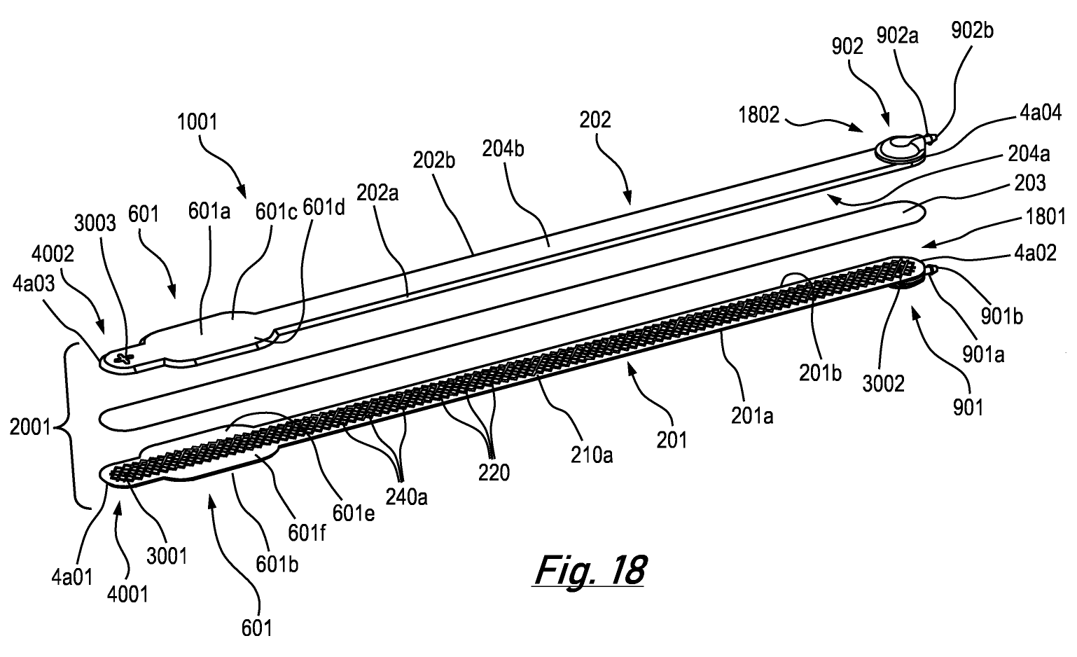

FIG. 17 is a photograph showing a plan view of part of an adaptor according to a further embodiment;

FIG. 18 is an exploded perspective view of an adaptor according to an even further embodiment;

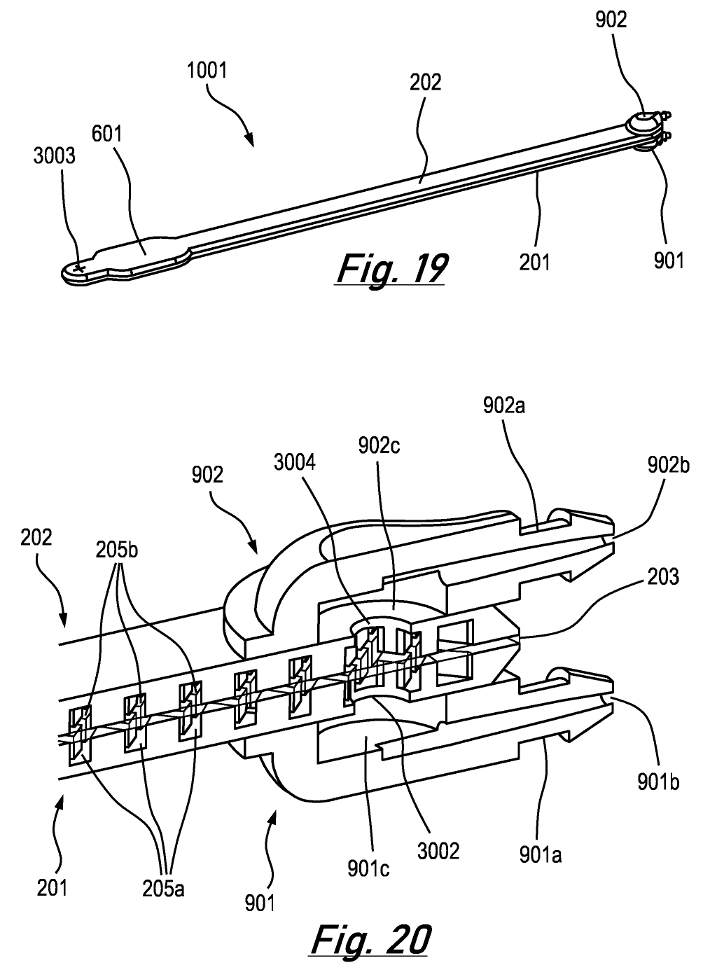

FIG. 19 is a perspective view of the adaptor shown in FIG. 18;

FIG. 20 is a perspective view of a cut-away of part of the adaptor shown in FIG. 18;

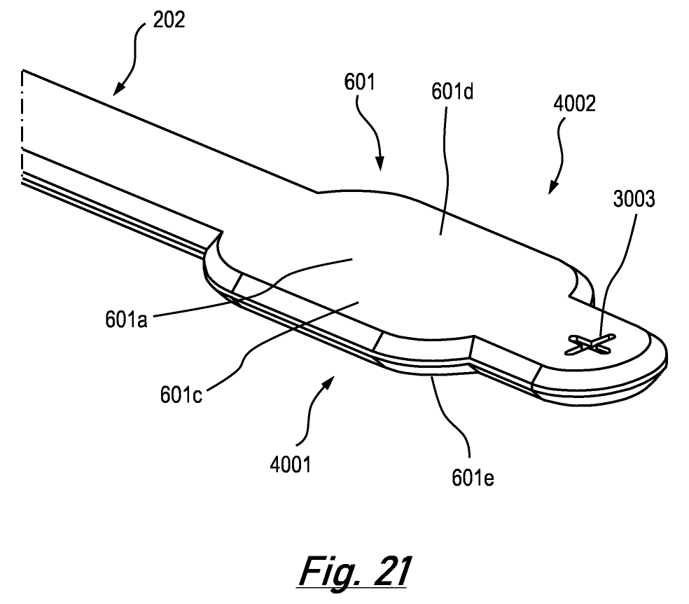

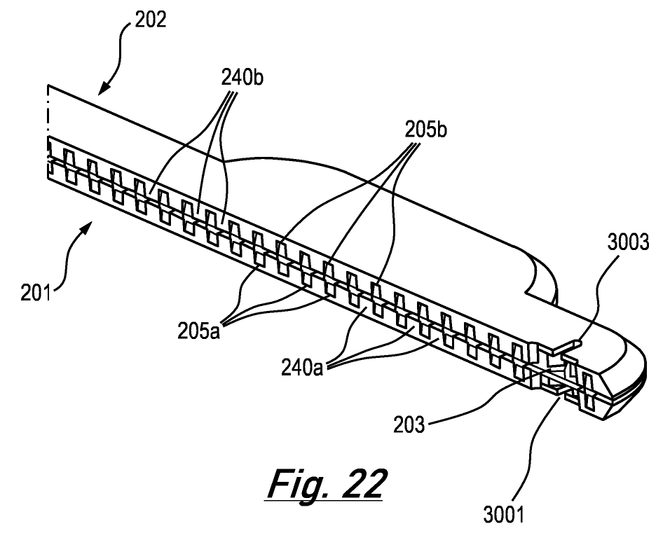

FIG. 21 is a perspective view of part of the adaptor of FIG. 18; and

FIG. 22 is a perspective view of a cut-away of part of the adaptor shown in FIG. 18.

Referring to FIGS. 1-6, an embodiment of an adaptor 1 is shown.

The adaptor 1 may be for adapting a surgical wound dressing for use in negative pressure wound therapy. The adaptor 1 may comprise an elongate, flexible airway 2. The airway 2 may be formed of a liquid impermeable material, for example a material selected from the group comprising polyurethane, thermoplastic polyurethane, thermoplastic elastomer, polyethylene, polypropylene, polyvinyl chloride, ethylene-vinyl acetate and a silicone material. In the described embodiment, the airway 2 is formed of polyurethane.

The airway 2 may have a maximum width which is greater than the maximum depth of the airway 2. The maximum depth of the airway 2 is measured as the distance from an uppermost surface 13 of the airway 2 to a lowermost surface 14 of the airway 2. In the described embodiment, the airway 2 has a maximum width which is at least four times the maximum depth of the airway 2.

The airway 2 may comprise a first end 4 and a second end (not shown in the embodiment of FIGS. 1-6), the second end being distal to the first end 4. In use, the airway 2 is arranged such that the first end 4 is proximal to a wound site of a patient. The airway may comprise an interior lumen 5 which may connect the first end 4 to the second end. As such, the airway 2 may be at least substantially hollow such that fluid can pass from the first end 4 to the second end, and vice versa. In particular, the interior lumen 5 may permit the flow of a gas from the first end 4 to the second end. The first end 4 may comprise a terminus 4a.

The second end may comprise a connector, for example a Luer or barb connector (not shown) which is connectable to a pump (not shown) (typically via a section of medical tubing, not shown). The second end may comprise at least one orifice for receiving the Luer or barb connector.

The first end 4 may comprise an opening 3. The opening 3 may be in communication with the interior lumen 5. The opening 3 may allow the interior lumen 5 to be in fluid communication with the exterior of the adaptor 1, in particular the exterior of the adaptor 1 which is proximal to the first end 4. In the described embodiment, the first end 4 may comprise a plurality of openings 3, in particular nine to twelve openings 3, such as twelve openings 3. Each opening 3 may be of a shape which is the same or different to any number of the other openings 3. For example, the shape of each opening may be a circle, square, rectangle, rhombus, triangle, pentagon, hexagon, heptagon, octagon, or in the shape of a cross or in the form of a slit. As shown in the described embodiment, each opening 3 may be a circle. Each opening may have a diameter, or length of its longest dimension, of no more than about 0.5cm, in particular and as shown in the described embodiment, about 0.1cm.

The airway 2 may comprise a sealing portion 6. The airway 2 comprises the sealing portion 6 toward its first end 4 but distal to the terminus 4a of the first end 4 relative to the location of the openings 3. As such, the openings 3 are arranged between the terminus 4a of the first end 4 and the sealing portion 6. The length of the airway 2 from its terminus 4a at the first end 4 to the sealing portion 6 may be no more than about 5cm. In the described embodiment, the length of the airway 2 from its terminus 4a to the sealing portion 6 is about 2cm.

In use, the sealing portion 6 provides a surface for adhering the adaptor 1 to an underside of a surgical wound dressing, such as to a peripheral adhesive skin contact layer of a surgical wound dressing. The sealing portion 6 also provides a surface for adhering the adaptor 1 to the peri wound skin of a patient.

The sealing portion 6 may comprise an upper sealing surface 7 and a lower sealing surface 8. In use, the upper sealing surface 7 may provide the surface for adhering the adaptor 1 to an underside of a surgical wound dressing, such as to a peripheral adhesive skin contact layer of a surgical wound dressing. In use, the lower sealing surface 8 may provide the surface for adhering the adaptor 1 to the peri wound skin of a patient. As such, the sealing portion 6 of the adaptor 1 is configured to provide a complete peripheral seal between the surgical wound dressing and the patient.

The lower sealing surface 8 may comprise an adhesive layer 9 which, prior to use, may be covered by a removable release liner 10. The removable release liner 10 may prevent accidental exposure of the adhesive layer 9 prior to use which may otherwise be detrimental to the adhesive property of the adhesive layer 9.

The sealing portion 6 may comprise a first wing 11 and a second wing 12. Each wing may extend outwardly from the airway 2 in a direction perpendicular to the length of the airway. As shown in FIG. 2, each wing 11, 12 is substantially of a semioval shape. Each wing 11, 12 extends outwardly a distance of about 3cm, such that the width of the sealing portion 6, that is the length from the outermost point of the first wing 11 to the outermost point of the second wing 12, may be about 6cm.

With particular reference to FIG. 6, the first wing 11 may comprise an upper sealing surface 7a and a lower sealing surface 8a. The second wing 12 may comprise an upper sealing surface 7b and a lower sealing surface 8b. The upper sealing surface 7a of the first wing 11 and the upper sealing surface 7b of the second wing 12 may join at the uppermost surface 13 of the airway 2. The lower sealing surface 8a of the first wing 11 and the lower sealing surface 8b of the second wing 12 may join at the lowermost surface 14 of the airway 2.

The adhesive layer 9 may extend across some, or all, of the lower sealing surfaces 8a, 8b. In the described embodiment, the adhesive layer extends across all of the lower sealing surfaces 8a, 8b.

As shown in the embodiment of FIG. 6, the upper sealing surface 7a of the first wing 11 extends outwardly from the uppermost surface 13 of the airway 2 and converges toward the lower sealing surface 8a. Also as shown in this embodiment, the upper sealing surface 7b of the second wing 12 extends outwardly from the uppermost surface 13 of the airway 2 and converges toward the lower sealing surface 8b.

In an alternate embodiment shown in FIG. 7, the arrangement of the airway 2 is much the same as in the embodiment shown in FIG. 6 but with a different configuration of the upper and lower surfaces of each wing. As such, among other features which are identical to the embodiment shown in FIG. 6, the adaptor 105 of the embodiment shown in FIG. 7 comprises a first wing 1105 and a second wing 1205. The first wing 1105 comprises an upper sealing surface 7a05 and a lower sealing surface 8a05. The upper sealing surface 7a05 extends outwardly from an uppermost surface 1305 of the airway. The second wing comprises an upper sealing surface 7b05 and a lower sealing surface 8b05. The upper sealing surface 7b05 extends outwardly from the uppermost surface 1305 of the airway. The lower sealing surfaces 8a05, 8b05 comprise an adhesive layer 905 which extends across both lower sealing surfaces 8a05, 8b05. Prior to use, the adhesive layer 905 may be covered by a removable release liner 1005. In contrast to the embodiment of FIG. 6, the lower sealing surfaces 8a05, 8b05 form a planar sealing surface which may facilitate adhesion to the peri wound skin of a patient when the adaptor 105 is in use. The upper sealing surface 7a05 of the first wing 1105 and the upper sealing surface 7b05 of the second wing 1205 each converge to the lower sealing surface 8a05 of the first wing 1105 and the lower sealing surface 8b05 of the second wing 1205, respectively. As such, the upper sealing surface 7a05 is convex relative to the planar lower sealing surface 8a05, and the upper sealing surface 7b05 is convex relative to the planar lower sealing surface 8b05.

In an alternate embodiment not shown in the figures, the upper sealing surfaces may form a planar sealing surface which may facilitate adhesion to an underside of a surgical wound dressing, in particular to a peripheral adhesive skin contact layer. In such an embodiment, the lower sealing surfaces may converge toward the respective upper sealing surfaces in much the same way as the upper sealing surfaces converge toward the planar, lower sealing surfaces in the embodiment shown in FIG. 7.

The adaptor 1 may have a length from the terminus 4a of the first end 4 to a terminus of the connector at the second end of about 30cm.

With reference to FIG. 8, there is shown the adaptor 1 of the embodiment shown in FIGS. 1 - 6 when used with a surgical wound dressing 16 to adapt the surgical wound dressing to be used in negative pressure wound therapy. The surgical wound dressing 16 may be a typical surgical wound dressing used for covering and protecting a wound. The dressing 16 may comprise a wound contact layer, a backing layer and at least one layer of absorbent material between the wound contact layer and the backing layer. The dressing may comprise a peripheral adhesive skin contact layer 15 in the form of a boundary of adhesive which is arranged around a wound in use. The peripheral adhesive skin contact layer 15 may form a window 17. The wound contact layer may be arranged in the window 17 and, therefore, can contact the wound in use to absorb exudate.

In use, the upper sealing surface 7, comprising sealings surfaces 7a and 7b, may provide a surface for adhering the adaptor 1 to a portion of the peripheral adhesive skin contact layer 15 of the underside of the surgical wound dressing 16. The lower sealing surface 8 may provide a surface for adhering the adaptor 1 to the peri wound skin of a patient. When preparing the adaptor 1 for use, the removable release liner 10 may be removed from the adhesive layer 9 to expose the adhesive layer 9 for adhesion to the peri wound skin of a patient. By adhering the adhesive layer 9 to the peri wound skin of a patient, and adhering a portion of the peripheral adhesive skin contact layer 15 of the wound dressing 16 to the upper sealing surfaces 7a, 7b, the sealing portion 6 of the adaptor 1 provides a complete peripheral seal between the surgical wound dressing 16 and the patient when in use. The openings 3 may therefore be arranged within the window 17 of the surgical dressing 16 between the wound contact layer and the wound.

As such, by way of an advantage of the present invention, the complete peripheral seal between the surgical wound dressing 16 and the patient provides a tight seal around the wound site and a portion of the first end 4 of the airway 2 which comprises the openings 3. The seal is preferably air-tight, or at least substantially air-tight, to allow for the efficient supply of negative pressure to the wound dressing to aid the wound-healing process. It will be appreciated by those skilled in the art that a completely air-tight seal is ideal, but with a sufficiently powerful pump, the seal need not be absolutely air-tight. As such, in use, the second end (not shown) of the adaptor may be connected, via a connector (not shown) and medical tubing (not shown) as herein described, to a pump (not shown). Activation of the pump can draw air from the wound and surrounding wound site within the confines of the peripheral seal, through the openings 3, along the interior lumen 5 toward the second end of the airway 2 and to the pump where the air may be expelled into the surrounding atmosphere.

Thus, the invention provides for adaptation of a non-negative pressure surgical wound dressing to a surgical wound dressing which can be used in negative pressure wound therapy which, advantageously, facilitates wound healing through a number of mechanisms, including removal of excess exudate, reduction in peri wound edema and increased perfusion. Combined with the physical forces exerted by the negative pressure which draw the wound edges together, this can result in improved wound outcomes. Moreover, the adaptor 1 of the invention allows for a negative pressure wound therapy system to be provided without using a negative pressure wound dressing. As such, the surgical wound dressings 16 which can be adapted, using the adaptor 1 of the invention, to provide negative pressure wound therapy do not comprise a port, or similar, in the backing layer of the wound dressing. It follows that the invention provides for a negative pressure wound therapy system without a wound dressing comprising a port. As such, the airway of the adaptor is not restricted in its orientation and the adaptor can be adhered to the surgical wound dressing in a position which is favourable for the patient, for example in a position where the adaptor, and therefore the airway, is not prone to being pressed against the skin of the patient or in a position where the patient is prone to obstructing the airway by resting upon the airway. Further, due to its absence, the relatively stiff suction port is not brought into close proximity with the wound site and therefore does not adversely affect the wound healing process, which could otherwise occur by, for example, the suction port being pressed against the wound which can cause a disturbance to the wound bed and damage the wound site, which may cause delays in healing of the wound site and discomfort for the patient.

A further embodiment of the invention is shown in FIGS. 9 -16.

With reference to FIGS. 9 -16, there is shown an adaptor 100. The adaptor 100 may be for adapting a surgical wound dressing for use in negative pressure wound therapy. The adaptor 100 may comprise an elongate, flexible airway 200. The airway 200 may be formed of a liquid impermeable material, for example a material selected from the group comprising polyurethane, thermoplastic polyurethane, thermoplastic elastomer, polyethylene, polypropylene, polyvinyl chloride, ethylene-vinyl acetate and a silicone material. In the described embodiment, the airway 200 is formed of translucent thermoplastic polyurethane, and may be formed by injection moulding.

The airway 200 may have a maximum width which is greater than the maximum depth of the airway 200. The airway 200 may have a maximum depth of from 0.5 mm to 2.0 mm. In the described embodiment, the airway 200 has a maximum depth of 1.5 mm. In the described embodiment, the airway 200 has a maximum width which is at least five times the maximum depth of the airway 200.

The airway 200 may comprise an elongated strip of material 20 comprising a pair of longitudinal edges arranged parallel to each other, except for at a sealing portion 60 and an inwardly tapered portion 181.

The airway 200 may comprise a first end 400 and a second end 180. The longitudinal edges are joined at the first end 400 by an outwardly curved portion, the outermost part of which forms the terminus 4a0 of the first end. The longitudinal edges may be joined by the inwardly tapered portion 181 at the second end 180. The second end 180 may be distal to the first end 400. The strip of material 20 may have a width (i.e., the distance between the first longitudinal edge and the second longitudinal edge) of about 15 mm and a length (i.e., the distance from the terminus 4a0 of the first end 400 to the terminus of the tapered portion 181) of about 300 mm.

The tapered portion 181 may comprise a tubular portion 182. The tubular portion 182 may extend away from the second end 180 to a greater extent than that of the tapered portion 181. The tubular portion 182 may comprise an orifice 183 for receiving a Luer or barb connector (not shown) which is connectable to a pump (not shown) typically via a section of medical tubing (not shown). The tubular portion 182 may have a depth of 0.8mm. As such, the tubular portion 182 may be of greater rigidity than any other portion of the airway 200 which facilitates insertion of the Luer or barb connector. The tubular portion 182 may extend for a length equal to less than 10%, for example about 6%, of the length of the airway 200. As shown in FIG. 9, the tubular portion 182 may extend into the airway 200 beyond that at which the strip of material 20 begins to taper at the second end 180.

The first end 400 may comprise an opening 300 in the shape of a cross. The opening 300 may be arranged central relative to the periphery of the first end 400.

The strip of material 20 may comprise an upwardly facing first surface 21 and a downwardly facing second surface. In use, the first surface 21 may face away from the wound site (not shown) and the second surface may face toward the wound site.

The first surface 21 of the strip of material 20 may comprise a grooved area comprising a plurality of grooves 22 and a plurality of projections 24. The grooved area may have a width which is equal to about 60% of the width of the strip of material 20, and a length which is equal to about 90% of the length of the strip of material 20. The grooved area may be substantially rectangular. At the second end 180 of the strip of material 20, the grooved area may terminate at substantially the same point at which the strip 20 begins to taper to form the tapered portion 181.

The plurality of grooves 22 may be arranged as an interlaced pattern of criss-crossed grooves 22, with each groove 22 being separated from another groove 22, or another portion of a groove 22, by a projection 24. Each projection 24 may extend from the base of a groove 22. Each groove 22 may have a width of 1 mm and a depth of 1.5 mm. By ‘width’ of a groove, we mean the distance between adjacent projections 24, between which is a groove 22. By ‘depth’ of a groove, we mean the distance between the uppermost surface or point of a projection 24 and the base of the groove 22 from which the projection 24 extends. In the embodiment shown in FIG. 9, each groove 22 has the same width and depth. The grooves may be formed by laser engraving/etching of the strip of material 20. The strip of material 20 may have a depth of 2.0 mm.

Each projection 24 may be arranged between each criss-crossed groove 22. In this embodiment, the projections 24 have a square cross-section when viewed in plan view.

The plurality of grooves 22 allow for the passage of air or another fluid along the airway 200. Each groove 22 may be in fluid communication with each other groove such that the plurality of grooves 22 forms an interconnected lattice of grooves in fluid communication with one another. Advantageously, the plurality of grooves 22 in this embodiment increases the overall volume of the grooves 22 of the airway 200. In turn, this means that a greater volume of fluid (for example air) is transmitted along the airway 200 at any given time compared to airways of the prior art which do not comprise a plurality of grooves, in particular an interlaced pattern of grooves. Further, when in use, the interconnected lattice of grooves 22 maintains a favourable flow rate of fluid along the airway 200 during compression or load upon the airway 200. As such, for example when used for transmitting a negative pressure to a wound site, the airway 200 of the invention maintains a negative pressure at a wound site even when the airway 200 has been folded and/or compressed by a user.

At least one groove 22 may be in fluid communication with the opening 300 at the first end 400, and with the tubular portion 182 at the second end 180. As such, in use as an adaptor 100 for adapting a surgical wound dressing for use in negative pressure wound therapy, when a pump (not shown) is connected to the tubular portion 182 (for example via a Luer or barb connector and medical tubing), and air is drawn toward the pump, the passage of air flows from the wound site, through the opening 300, along the grooves 22 and through the tubular portion 182 toward the pump. As shown in FIG. 9 at least, each groove 22 is in fluid communication with each other groove 22. As such, the opening 300 and the tubular portion 182 need only be in fluid communication with at least one groove 22 in order to allow the passage of air along all grooves 22.

The airway 200 may comprise a sealing portion 60. The airway 200 may comprise the sealing portion 60 toward its first end 400 but distal to the terminus 4a0 of the first end 400 relative to the location of the opening 300. As such, the opening 300 may be arranged between the terminus 4a0 of the first end 400 and the sealing portion 60. The length of the airway 200 from its terminus 4a0 at the first end 400 to the sealing portion 60 may be no more than about 5cm. In the described embodiment, the length of the airway 200 from its terminus 4a0 to the sealing portion 60 is about 2cm.

In use, the sealing portion 60 provides a surface for adhering the adaptor 100 to an underside of a surgical wound dressing, such as to a peripheral adhesive skin contact layer of a surgical wound dressing. The sealing portion 60 also provides a surface for adhering the adaptor 100 to the peri wound skin of a patient.

The sealing portion 60 may comprise an upper sealing surface and a lower sealing surface. In use, the upper sealing surface may provide the surface for adhering the adaptor 100 to an underside of a surgical wound dressing, such as to a peripheral adhesive skin contact layer of a surgical wound dressing. In use, the lower sealing surface may provide the surface for adhering the adaptor 100 to the peri wound skin of a patient. As such, the sealing portion 60 of the adaptor 100 is configured to provide a complete peripheral seal between the surgical wound dressing and the patient.

The lower sealing surface may comprise an adhesive layer 900 which, prior to use, may be covered by a removable release liner 101. The removable release liner 101 may prevent accidental exposure of the adhesive layer 900 prior to use which may otherwise be detrimental to the adhesive property of the adhesive layer 900.

The sealing portion 60 may comprise a first wing 110 and a second wing 120. Each wing 110, 120 may extend outwardly from the airway 200 in a direction perpendicular to the length of the airway 200. Each wing 110, 120 extends outwardly from its respective longitudinal edge of the airway 200 a distance of about 1.5cm, such that the width of the sealing portion 60, that is the length from the outermost point of the first wing 110 to the outermost point of the second wing 120, may be about 5cm. Each wing curves outwardly, from its respective longitudinal edge of the airway 200, to a distance of about 1.5cm from the longitudinal edge, and then extends linearly to the length of the airway 200, such that the linear portions of each wing 110, 120 are parallel to one another, for a distance of about 3cm, and then curves toward the respective longitudinal edge of the airway 200.

The first wing 110 may comprise an upper sealing surface 7a0 and a lower sealing surface 8a0. The second wing 120 may comprise an upper sealing surface 7b0 and a lower sealing surface 8b0. The upper sealing surface 7a0 of the first wing 110 and the upper sealing surface 7b0 of the second wing 120 may be substantially planar to provide a substantially flat upper surface to the airway 200. As shown in FIG. 13, the lower sealing surface 8a0 of the first wing 110 and the lower sealing surface 8b0 of the second wing 120 may each upwardly curve toward the upper sealing surface 7a0 and the upper sealing surface 7b0, respectively.

As shown in FIG. 14, the adhesive layer 900 may extend across all of the lower sealing surfaces 8a0, 8b0.

A cover in the form of a film 23 may be adhered to the first surface 21 of the strip of material 20. The film 23 may have substantially the same width and length as that of the strip of material 20 to prevent overlapping edges of either the strip of material 20 or the film 23. The film 23 may be adhered to the strip of material 20 at its longitudinal edges. The film 23 may be adhered to the strip of material 20 by heat lamination. The film 23 may be formed of transparent polyurethane. The film 23 may have a thickness of about 0.2mm.

Once the film 23 is adhered to the strip of material 20, the plurality of grooves 22 form a plurality of channels 25 between the strip 20 and the film 23. Each channel of the plurality of channels 25 may be in fluid communication with each other channel such that the plurality of channels 25 forms a network of interconnected channels in fluid communication with one another. The interconnected channels 25 may be in fluid communication with the orifice 183 of the tubular portion 182 at the second end 180, and with opening 300 at the first end 400. The plurality of channels 25 allow for the passage of air to or from the wound site via a pump, for example used as part of a negative pressure wound dressing system. Compression of the film 23 toward the strip of material 20 is prevented, or at least significantly limited, by the plurality of projections 25 arranged along the grooved area.

Compression of the film 23 toward the strip of material 20 is also prevented, or at least significantly limited, by the relatively small width of the plurality of grooves 22 such that when a typical load exerts pressure upon the film 23, the film 23 does not compress into the grooves 22, or at least compresses into a portion of the grooves 22 only, so that the flow of fluid along the grooves 22 is not prevented. In use, when a negative pressure is applied to the wound site and air is drawn away from the wound site along the plurality of channels 25, the film 23 will compress downwardly, toward the channels 25. However, the film 23 does not obstruct the channels 25 due to the plurality of projections 24 upon which the film 23 compresses. As such, the film 23 does not collapse into the channels 25, therefore, the described arrangement allows for the unobstructed passage of air along the channels 25.

The film 23 may be substantially planar.

It will be understood that the dimensions (depth, width and length) of each channel 25 will be substantially the same as the corresponding dimensions of each groove 22.

With reference to FIGS. 15 and 16, there is shown the adaptor 100 of the embodiment shown in FIGS. 9 - 16 when used with a surgical wound dressing 160 to adapt the surgical wound dressing 160 to be used in negative pressure wound therapy. The surgical wound dressing 160 may be a typical surgical wound dressing used for covering and protecting a wound. The dressing 160 may comprise a wound contact layer 26, a backing layer and at least one layer of absorbent material between the wound contact layer 26 and the backing layer. The dressing 160 may comprise a peripheral adhesive skin contact layer 150 in the form of a boundary of adhesive which is arranged around a wound in use. The peripheral adhesive skin contact layer 150 may form a window. The wound contact layer 26 may be arranged in the window and, therefore, can contact the wound in use to absorb exudate.

In use, the upper sealing surfaces 7a0 and 7b0, may provide a surface for adhering the adaptor 100 to a portion of the peripheral adhesive skin contact layer 150 of the underside 27 of the surgical wound dressing 160. The lower sealing surfaces 8a0, 8b0 may provide a surface for adhering the adaptor 100 to the peri wound skin of a patient.

When preparing the adaptor 100 for use, the removable release liner 101 may be removed from the adhesive layer 900 to expose the adhesive layer 900 for adhesion to the peri wound skin of a patient. By adhering the adhesive layer 900 to the peri wound skin of a patient, and adhering the upper sealing surfaces 7a0 and 7b0 to a portion of the peripheral adhesive skin contact layer 150 on the underside 27 of the wound dressing 160, the sealing portion 60 of the adaptor 100 provides a complete peripheral seal between the surgical wound dressing 160 and the patient when in use. The opening 300 may therefore be arranged within the window of the surgical dressing 160 between the wound contact layer 26 and the wound.

As such, by way of an advantage of the present invention, the complete peripheral seal between the surgical wound dressing 160 and the patient provides a tight seal around the wound site and a portion of the first end 400 of the airway 200 which comprises the opening 300. The seal is preferably air-tight, or at least substantially air-tight, to allow for the efficient supply of negative pressure to the wound dressing to aid the wound-healing process. It will be appreciated by those skilled in the art that a completely air-tight seal is ideal, but with a sufficiently powerful pump, the seal need not be absolutely air-tight. As such, in use, the second end 180 of the adaptor 200 may be connected, via a connector (not shown) attached to the tubular portion 182, and medical tubing (not shown), to a pump (not shown). Activation of the pump can draw air from the wound and surrounding wound site within the confines of the peripheral seal, through the opening 300, along the channels 25 toward the second end 180 of the airway 200 and to the pump where the air may be expelled into the surrounding atmosphere.

Thus, the invention provides for adaptation of a non-negative pressure surgical wound dressing to a surgical wound dressing which can be used in negative pressure wound therapy which, advantageously, facilitates wound healing through a number of mechanisms, including removal of excess exudate, reduction in peri wound edema and increased perfusion. Combined with the physical forces exerted by the negative pressure which draw the wound edges together, this can result in improved wound outcomes. Moreover, the adaptor 100 of the invention allows for a negative pressure wound therapy system to be provided without using a negative pressure wound dressing. As such, the surgical wound dressings 160 which can be adapted, using the adaptor 100 of the invention, to provide negative pressure wound therapy do not comprise a port, or similar, in the backing layer of the wound dressing 160. It follows that the invention provides for a negative pressure wound therapy system without a wound dressing comprising a port. As such, the airway 200 of the adaptor 100 is not restricted in its orientation and the adaptor 100 can be adhered to the surgical wound dressing 160 in a position which is favourable for the patient, for example in a position where the adaptor 100, and therefore the airway 200, is not prone to being pressed against the skin of the patient or in a position where the patient is prone to obstructing the airway 200 by resting upon the airway 200. Further, due to its absence, the relatively stiff suction port is not brought into close proximity with the wound site and therefore does not adversely affect the wound healing process, which could otherwise occur by, for example, the suction port being pressed against the wound which can cause a disturbance to the wound bed and damage the wound site, which may cause delays in healing of the wound site and discomfort for the patient.

With reference to FIG. 17, there is shown part of an airway 2000 of a further embodiment of an adaptor 1000.

The adaptor 1000 comprises an airway 2000. The airway 2000 may comprise an elongated strip of material 250 comprising longitudinal edges and a first surface 250a and a second surface (not shown). Many of the features of the adaptor 1000 are identical to those of adaptor 100 of the embodiment described above. However, adaptor 1000 differs from adaptor 100 in that the adaptor 1000 may comprise an airway 2000 comprising two distinct, separated channelled areas 251, 252. The airway 2000 may comprise a first channelled area 251 and a second channelled area 252. The first channelled area 251 may be separated from the second channelled area 252 by a seal 253 formed between the film (not shown) and the strip of material 250. The first channelled area 251 may not be in fluid communication with the second channelled area 252. The seal 253 may be formed by heat lamination. The first channelled area 251 may comprise two criss-crossing channels 251a, and the second channelled area 252 may comprise two criss-crossing channels 252a. Each channelled area 251, 252 may be formed by the combination of grooves and projections as in the previously mentioned embodiments. In the first channelled area 251, the channels 251a are separated by a plurality of projections 241. In the second channelled area 252, the channels 252a are separated by a plurality of projections 242. Advantageously, in this embodiment, fluid can flow toward a wound site via the channels 251a in the first channelled area 251, and fluid can simultaneously flow away from a wound site via the channels 252a in the second channelled area 252.

With reference to FIGS. 18 -22, there is shown an adaptor 1001 of a further embodiment of the invention. The adaptor 1001 may be for adapting a surgical wound dressing for use in negative pressure wound therapy. The adaptor 1001 may comprise an elongate, flexible airway 2001. The airway 2001 may be formed of a liquid impermeable material, for example a material selected from the group comprising polyurethane, thermoplastic polyurethane, thermoplastic elastomer, polyethylene, polypropylene, polyvinyl chloride, ethylene-vinyl acetate and a silicone material. In the described embodiment, the airway 2001 is formed of translucent thermoplastic polyurethane, and may be formed by injection moulding.

The airway 2001 may have a maximum width which is greater than the maximum depth of the airway 2001. The airway 2001 may have a maximum depth of from 0.5 mm to 2.0 mm. In the described embodiment, the airway 2001 has a maximum depth of 1.5 mm. In the described embodiment, the airway 2001 has a maximum width which is at least five times the maximum depth of the airway 2001.

The airway 2001 may comprise a first elongated strip of material 201 comprising a pair of longitudinal edges 201a, 201b.

The first elongated strip of material 201 may comprise a first end 4001 and a second end 1801. The longitudinal edges 201a, 201b may be joined at the first end 4001 by an outwardly curved portion, the outermost part of which forms the terminus 4a01 of the first end. The longitudinal edges 201a, 201b may be joined by an outwardly curved portion, the outermost part of which forms the terminus 4a02 of the second end 1801. The second end 1801 may be distal to the first end 4001. The strip of material 201 may have a width (i.e., the distance between the first longitudinal edge 201a and the second longitudinal edge 201b) of about 15 mm and a length (i.e., the distance from the terminus 4a01 of the first end 4001 to the terminus 4a02 of the second end 1801) of about 300 mm.

The first end 4001 may comprise a first opening 3001 in the shape of a cross. The first opening 3001 may be arranged central relative to the periphery of the first end 4001.

The strip of material 201 may comprise an upwardly facing first surface 210a and a downwardly facing second surface. In use, the first surface 210a may face away from the wound site (not shown) and the second surface may face toward the wound site.

The first surface 210a of the strip of material 201 may comprise a grooved area comprising a plurality of grooves 220 and a plurality of projections 240a. The grooved area may have a width which is equal to about 60% of the width of the strip of material 201, and a length which is equal to about 90% of the length of the strip of material 201. The grooved area may be substantially rectangular.

The plurality of grooves 220 may be arranged as an interlaced pattern of criss-crossed grooves 220, with each groove 220 being separated from another groove 220, or another portion of a groove 220, by a projection 240a. Each projection 240a may extend from the base of a groove 220. Each groove 220 may have a width of 1 mm and a depth of 1.5 mm. The grooves may be formed by laser engraving/etching of the strip of material 201. The strip of material 201 may have a depth of 2.0 mm.

The plurality of grooves 220 allow for the passage of air or another fluid along the airway 2001. Each groove 220 may be in fluid communication with each other groove 220 such that the plurality of grooves 220 forms an interconnected lattice of grooves in fluid communication with one another. Advantageously, the plurality of grooves 220 in this embodiment increases the overall volume of the grooves 220 of the airway 2001. In turn, this means that a greater volume of fluid (for example air) is transmitted along the airway 2001 at any given time compared to known airways of the prior art which do not comprise a plurality of grooves, in particular an interlaced pattern of grooves. Further, when in use, the interconnected lattice of grooves 220 maintains a favourable flow rate of fluid along the airway 2001 during compression or load upon the airway 2001. As such, for example when used for transmitting a negative pressure to a wound site, the airway 2001 of the invention maintains a negative pressure at a wound site even when the airway 2001 has been folded and/or compressed by a user.

The second end 1801 of the first strip of material 201 may comprise a second opening 3002 in the shape of a cross. The second opening 3002 may be arranged central relative to the periphery of the second end 1801.

At least one groove 220 may be in fluid communication with the first opening 3001 at the first end 4001, and with the second opening 3002 at the second end 1801.

The airway 2001 may comprise a film 203. The film 203 may be adhered to the first surface 210a of the first strip of material 201. The film 203 may have substantially the same width and length as that of the strip of material 201 to prevent overlapping edges of either the strip of material 201 or the film 203. The film 203 may be adhered to the strip of material 201 at its longitudinal edges. The film 203 may be adhered to the strip of material 201 by heat lamination. The film 203 may be formed of transparent or translucent polyurethane. The film 203 may have a thickness of about 0.2mm.

Once the film 203 is adhered to the first strip of material 201, the plurality of grooves 220 form a first plurality of channels 205a between the first strip of material 201 and the film 203. Each channel of the first plurality of channels 205a may be in fluid communication with each other channel such that the first plurality of channels 205a forms a network of interconnected channels in fluid communication with one another. The interconnected channels 205a may be in fluid communication with the first opening 3001 at the first end 4001 and with the second opening 3002 at the second end 1801.

The first plurality of channels 205a allow for the passage of air to or from the wound site via a pump, for example used as part of a negative pressure wound dressing system. Compression of the film 203 toward the strip of material 201 may be prevented, or at least significantly limited, by the plurality of projections 240a arranged along the grooved area.

Compression of the film 203 toward the strip of material 201 may also be prevented, or at least significantly limited, by the relatively small width of the plurality of grooves 220 such that when a typical load exerts pressure upon the film 203, the film 203 does not compress into the grooves 220 so that the flow of fluid along the grooves 220 is not prevented.

The film 203 may be substantially planar.

The airway 2001 may comprise a second elongated strip of material 202.

The second elongated strip of material 202 may comprise a first end 4002 and a second end 1802. The second elongated strip of material 202 may comprise a pair of longitudinal edges 202a, 202b. The longitudinal edges 202a, 202b may be joined at the first end 4002 by an outwardly curved portion, the outermost part of which forms the terminus 4a03 of the first end. The longitudinal edges 202a, 202b may be joined by an outwardly curved portion, the outermost part of which forms the terminus 4a04 of the second end 1802. The second end 1802 may be distal to the first end 4002. The second strip of material 202 may have a width (i.e., the distance between the first longitudinal edge 202a and the second longitudinal edge 202b) of about 15 mm and a length (i.e., the distance from the terminus 4a03 of the first end 4002 to the terminus 4a04 of the second end 1802) of about 300 mm.

The first end 4002 of the second strip of material 202 may comprise a third opening 3003 in the shape of a cross. The third opening 3003 may be arranged central relative to the periphery of the first end 4002.

The second strip of material 202 may comprise a downwardly facing first surface 204a and an upwardly facing second surface 204b. In use, the first surface 204a may face toward the wound site (not shown) and the second surface 204b may face away from the wound site.

The first surface 204a of the second strip of material 202 may comprise a grooved area comprising a plurality of grooves (not shown) and a plurality of projections 240b. The grooved area of the first surface 204a of the second strip of material 202 may comprise each of the features of the grooved area of the first surface 210a of the first strip of material 201. The grooved area may have a width which is equal to about 60% of the width of the second strip of material 202, and a length which is equal to about 90% of the length of the second strip of material 202. The grooved area may be substantially rectangular.

The plurality of grooves may be arranged as an interlaced pattern of criss-crossed grooves, with each groove being separated from another groove, or another portion of a groove, by a projection 240b. Each projection 240b may extend from the base of a groove. Each groove may have a width of 1 mm and a depth of 1.5 mm. The grooves may be formed by laser engraving/etching of the strip of material 202. The strip of material 202 may have a depth of 2.0 mm.

Each groove may be in fluid communication with each other groove such that the plurality of grooves forms an interconnected lattice of grooves in fluid communication with one another. Advantageously, the plurality of grooves in this embodiment increases the overall volume of the grooves of the airway 2001. In turn, this means that a greater volume of fluid (for example air) is transmitted along the airway 2001 at any given time compared to airways of the prior art which do not comprise a plurality of grooves, in particular an interlaced pattern of grooves. Further, when in use, the interconnected lattice of grooves maintains a favourable flow rate of fluid along the airway 2001 during compression or load upon the airway 2001. As such, for example when used for transmitting a negative pressure to a wound site, the airway 2001 of the invention maintains a negative pressure at a wound site even when the airway 2001 has been folded and/or compressed by a user.

The second end 1802 may comprise a fourth opening 3004 in the shape of a cross. The fourth opening 3004 may be arranged central relative to the periphery of the second end 1802.

At least one groove may be in fluid communication with the third opening 3003 at the first end 4002 of the second strip of material 202, and with the fourth opening 3004 at the second end 1802 of the second strip of material 202.

The airway 2001 may comprise the second strip of material 202 such that first surface 204a of the second strip 202 is arranged over the film 203. The first surface 204a of the second strip 202 may be adhered to the film 203 (for example by an adhesive, alternatively heat lamination may be used) about its longitudinal edges 202a, 202b. Once the second strip of material 202 is adhered to the film 203, the plurality of grooves form a second plurality of channels 205b between the second strip of material 202 and the film 203. Each channel of the second plurality of channels 205b may be in fluid communication with each other channel such that the second plurality of channels 205b forms a network of interconnected channels in fluid communication with one another. The interconnected channels 205b may be in fluid communication with the third opening 3003 at the first end 4002 and with the fourth opening 3004 at the second end 1802.

The peripheral dimensions of the second strip of material 202 may be substantially equal to the peripheral dimensions of the first strip of material 201.

The plurality of channels 205b may allow for the passage of air to or from the wound site via a pump, for example used as part of a negative pressure wound dressing system. Compression of the film 203 toward the strip of material 202 may be prevented, or at least significantly limited, by the plurality of projections 240b arranged along the grooved area.

Compression of the film 203 toward the strip of material 202 is also prevented, or at least significantly limited, by the relatively small width of the plurality of grooves such that when a typical load exerts pressure upon the film 203, the film 203 does not compress into the grooves, or at least compresses into a portion of the grooves only, so that the flow of fluid along the grooves is not prevented.

In the described embodiment, the first plurality of channels 205a formed between the first strip of material 201 and the film 203 are not in fluid communication with the second plurality of channels 205b formed between the second strip of material 202 and the film 203. The first opening 3001 comprised in the first strip 201 and the third opening 3003 comprised in the second strip 202 may not be in fluid communication with one another, and may be separated from one another by the film 203.

The second opening 3002 comprised in the first strip 201 and the fourth opening 3004 comprised in the second strip 202 may not be in fluid communication with one another, and may be separated from one another by the film 203.

The airway 2001 may comprise a sealing portion 601. The airway 2001 may comprise the sealing portion 601 toward its first end 4001, 4002 but distal to the terminus 4a01, 4a03 of the first end 4001, 4002 relative to the location of the openings 3001, 3003. As such, the openings 3001, 3003 may be arranged between the terminus 4a01, 4a03 of the first end 4001, 4002 and the sealing portion 601. The length of the airway 2001 from its terminus 4a01, 4a03 at the first end 4001, 4002 to the sealing portion 601 may be no more than about 5cm. In the described embodiment, the length of the airway 2001 from its terminus 4a01, 4a03 to the sealing portion 601 is about 2cm.

In use, the sealing portion 601 provides a surface for adhering the adaptor 1001 to an underside of a surgical wound dressing, such as to a peripheral adhesive skin contact layer of a surgical wound dressing. The sealing portion 601 also provides a surface for adhering the adaptor 1001 to the peri wound skin of a patient.

The sealing portion 601 may comprise an upper sealing surface 601a and a lower sealing surface 601b. In use, the upper sealing surface 601a may provide the surface for adhering the adaptor 1001 to an underside of a surgical wound dressing, such as to a peripheral adhesive skin contact layer of a surgical wound dressing. In use, the lower sealing surface 601b may provide the surface for adhering the adaptor 1001 to the peri wound skin of a patient. As such, the sealing portion 601 of the adaptor 1001 is configured to provide a complete peripheral seal between the surgical wound dressing and the patient.

The lower sealing surface 601b may comprise an adhesive layer (not shown) which, prior to use, may be covered by a removable release liner (not shown). The removable release liner may prevent accidental exposure of the adhesive layer prior to use which may otherwise be detrimental to the adhesive property of the adhesive layer.

The upper sealing surface 601a may comprise a first wing 601c and a second wing 601d. The lower sealing surface 601b may comprise a third wing 601e and a fourth wing 601f. Each wing 601c, 601d, 601e, 601f may extend outwardly from the airway 2001 in a direction perpendicular to the length of the airway 2001. Each wing 601c, 601d, 601e, 601f may extend outwardly from its respective longitudinal edge of the airway 2001 a distance of about 1.5cm, such that the width of the sealing portion 601, that is the length from the outermost point of the first wing 601c and third wing 601e to the outermost point of the second wing 601d and fourth wing 601f, may be about 5cm. Each wing curves outwardly, from its respective longitudinal edge of the airway 2001, to a distance of about 1.5cm from the longitudinal edge, and then extends linearly to the length of the airway 2001, such that the linear portion of the first wing 601c is parallel with the linear portion of the second wing 601d, and the linear portion of the third wing 601e is parallel with the linear portion of the fourth wing 601f, for a distance of about 3cm, and then each wing curves toward the respective longitudinal edge of the airway 2001.

An upper surface of the first wing 601c and second wing 601d may comprise a portion of the upper sealing surface 601a. A lower surface of the third wing 601e and the fourth wing 601f may comprise a portion of the lower sealing surface 601b. The upper sealing surface 601a may be substantially planar to provide a substantially flat upper surface to the airway 2001. The lower sealing surface 601b may be substantially planar to provide a substantially flat lower surface to the airway 2001.

The second end 1801 of the first strip of material 201 may comprise a first connector 901 adhered to its second surface. The first connector 901 may comprise a nozzle 901a having an aperture 901b, and a surface comprising an orifice 901c arranged in fluid communication with the aperture 901b. The first connector 901 may be arranged such that the orifice 901c is in fluid communication with the second opening 3002 at the second end 1801 of the first strip 201. Thus, the first plurality of channels 205a may be in fluid communication with the orifice 901c of the first connector 901, and also, therefore, the aperture of the nozzle 901b.

The second end 1802 of the second strip of material 202 may comprise a second connector 902 adhered to its upwardly facing second surface 204b. The second connector 902 may comprise a nozzle 902a having an aperture 902b, and a surface comprising an orifice 902c in fluid communication with the aperture 902b. The second connector 902 may be arranged such that the orifice 902c is in fluid communication with the fourth opening 3004 at the second end 1802 of the second strip of material 202. Thus, the second plurality of channels 205b may be in fluid communication with the orifice 902c of the second connector 902, and therefore, the aperture of the nozzle 902b.

A pump (not shown) may be connected to one or each of the first and second connectors 901, 902 (for example via a Luer or barb connector and medical tubing).

As such, in use as an adaptor 1001 for adapting a surgical wound dressing for use in negative pressure wound therapy, a pump (not shown) may be connected to the first connector 901 (for example via a Luer or barb connector and medical tubing), and air may be drawn toward the pump. The passage of air may flow from the wound site, through the first opening 3001, along the first plurality of channels 205a and through the second opening 3002, through the orifice 901c of the first connector 901, along the nozzle 901a, through the aperture 901b, and along any medical tubing toward the pump.

Further, the embodiment shown in FIGS. 18-22 allows for a fluid (for example air) to be delivered to the wound site simultaneously to the drawing of air from the wound site as described above. For example, a pump (not shown) may be connected to the second connector 902 (for example via a Luer or barb connector and medical tubing), and air may be delivered from the pump toward the wound site. The passage of air may flow from the pump, along any medical tubing, through the aperture 902b of the nozzle 902a of the second connector 902, through the orifice 902c of the second connector 902, and through the fourth opening 3004, along the second plurality of channels 205b and through the third opening 3003, toward the wound site.

In a further embodiment of the invention, which is not shown in the Figures, the airway may comprise a transmission layer, a top layer and a bottom layer. The top layer may be provided over the transmission layer. The bottom layer may be provided below the transmission layer. The top layer and the bottom layer may enclose the transmission layer, or at least a significant portion of the transmission layer. The transmission layer may be elongated and substantially rectangular when viewed in plan view. The transmission layer may be substantially planar.

The transmission layer may comprise an upper layer and a lower layer, between which is a space. The transmission layer may be a 3D spacer fabric. The 3D spacer fabric may be formed of polyester and may be knitted, being formed by warp knitting. The 3D spacer fabric may have a basis weight of between 200 and 250 g/m², preferably 225 g/m². The 3D spacer fabric may have a thickness (i.e., the distance between the upper layer and the lower layer) of 2.75mm.

The space may comprise a plurality of monofilaments separating the upper layer and the lower layer. Each monofilament may be joined to an inner surface of the upper layer of the 3D spacer fabric transmission layer and to an inner surface of the lower layer of the 3D spacer fabric transmission layer. Each of the plurality of monofilaments may be formed of polyester.

The top layer and the bottom layer of the airway may each be constructed from a liquid impermeable material. In this embodiment, the top layer and bottom layer are each a polyurethane film. The top layer and the bottom layer may each be waterproof, gas impermeable and moisture vapour permeable. Each of the top layer and the bottom layer may have a thickness of 0.2mm. The top layer may be adhered to the bottom layer around substantially all of its periphery to form a substantially rectangular elongate channel holding the transmission layer therein. The top layer may, for example, be adhered to the bottom layer by heat lamination. The top layer may not be adhered to the bottom layer at its periphery at a first end of the airway, such that an opening is formed between the top layer and the bottom layer. Alternatively, the top layer may be adhered to the bottom layer around its periphery, including at the first end of the airway, and at least one opening may be formed in the first end such that the space substantially enclosed by the top layer and the bottom layer is in fluid communication with the opening.

The airway of this embodiment may comprise a sealing portion having the features of the sealing portion of the first embodiment described herein. As such, the airway may comprise a first wing and a second wing. The first wing may comprise an upper sealing surface which extends outwardly from the top layer of the airway, and a lower sealing surface which extends outwardly from the bottom layer of the airway. The upper and lower sealing surfaces may converge to each other. The second wing may comprise an upper sealing surface which extends outwardly from the top layer of the airway, and a lower sealing surface which extends outwardly from the bottom layer of the airway. The upper and lower sealing surfaces may converge to each other. The first wing and the second wing may be formed of the same material as the airway. The lower sealing surfaces may comprise an adhesive layer covered by a removable release liner.

In use, the upper sealing surfaces may be adhered to a portion of a peripheral adhesive skin contact layer of a surgical wound dressing. The release liner may be removed from the adhesive layer of the lower sealing surfaces of the sealing portion and adhered to the peri wound skin of a patient. The opening at the first end of the airway may be arranged within the window of the peripheral adhesive skin contact layer, proximal to the wound site such that the space substantially enclosed by the top and bottom layers is in communication with the openings and therefore the wound site.

The embodiment therefore provides a complete peripheral seal between the surgical wound dressing and the patient, forming a tight seal around the wound site and a portion of the first end of the airway which comprises the opening. As such, in use, a second end of the adaptor may be connected, via a connector and medical tubing to a pump. Activation of the pump can draw air from the wound and surrounding wound site, through the opening, along the space enclosed by the top and bottom layers of the airway, toward the second end of the airway and to the pump where the air is expelled into the surrounding atmosphere. Thus, the invention provides for adaptation of a non-negative pressure surgical wound dressing to a surgical wound dressing which can be used in negative pressure wound therapy which, advantageously, facilitates wound healing through a number of mechanisms, including removal of excess exudate, reduction in peri wound edema and increased perfusion. Combined with the physical forces exerted by the negative pressure which draw the wound edges together, this can result in improved wound outcomes.

The one or more embodiments are described above by way of example only. Many variations are possible without departing from the scope of protection afforded by the appended claims.

1. An adaptor for adapting a surgical wound dressing for use in negative pressure wound therapy,
the adaptor comprising an elongate flexible airway having an opening at a first end; a connector at an opposing second end for connection to medical tubing and an interior lumen therebetween;
the airway comprising a sealing portion having an upper sealing surface and a lower sealing surface, wherein the lower sealing surface of the sealing portion comprises an adhesive layer and a removable release liner on the adhesive layer, the upper sealing surface of the sealing portion being configured, in use, to be adhered to a peripheral adhesive skin contact layer on the underside of the surgical wound dressing and the lower sealing surface being configured in use to be adhered to the skin of a patient to complete a peripheral seal between the surgical wound dressing and the patient.

2. An adaptor according to claim 1, wherein the airway is formed of a material selected from the group comprising polyurethane, thermoplastic polyurethane, thermoplastic elastomer, polyethylene, polypropylene, polyvinyl chloride, ethylene-vinyl acetate and a silicone material.

3. An adaptor according to claim 1 or 2 , wherein the airway comprises a transmission layer, a top layer and a bottom layer, wherein the top layer is provided over the transmission layer and the bottom layer is provided below the transmission layer.

4. An adaptor according to any of claims 1-3 , wherein the airway comprises a first strip of material having a first surface comprising at least one groove, the airway further comprising a film arranged over the first surface of the first strip of material and adhered to the first strip of material around its longitudinal edges, wherein the at least one groove is provided with at least one projection to form at least two channels between the first strip and the film.

5. An adaptor according to claim 4, wherein the at least one groove is provided with a plurality of projections, such that there is provided a plurality of channels between the first strip and the film.

6. An adaptor according to claim 5, wherein the channels form a network of interconnected channels.

7. An adaptor according to any of claims 4-6, wherein the at least two channels are in fluid communication with the opening.

8. An adaptor according to any of claims 4-7, wherein the or each projection extends from a base of the or each groove.

9. An adaptor according to any of claims 4-8, wherein the or each projection extends to a height equal to, or at least substantially equal to, the height of the first strip of material.

10. An adaptor according to any of claims 4-9, wherein the or each projection comprises an upper surface which is adhered to a lower surface of the film.

11. An adaptor according to any of claims 4-10 , wherein the or each groove has a depth of at least 0.5mm.

12. An adaptor according to any of claims 4-11 , wherein the at least two channels comprises at least a first channelled area and a second channelled area, wherein the first channelled area comprises at least a first channel, and the second channelled area comprises at least a second channel, wherein the first channel is not in fluid communication with the second channel.

13. An adaptor according to claim 12, wherein the first channel and the second channel are separated by a seal between the strip and the film.

14. An adaptor according to any of claims 4-11 , wherein the airway comprises a second strip of material having a first surface comprising at least one groove provided with at least one projection, the first surface of the second strip of material being arranged over the film and adhered to the film around its longitudinal edges to form at least two channels, between the second strip and the film.

15. An adaptor according to claim 14, wherein the at least two channels between the first strip and the film are not in fluid communication with the at least two channels between the second strip and the film.

16. An adaptor according to claim 14 or 15 , wherein the connector is a first connector for connection of the at least two channels between the first strip and the film to a first medical tubing and an interior lumen therebetween.

17. An adaptor according to any of claims 14 -16 , wherein the airway comprises a second connector for connection of the at least two channels between the second strip and the film to a second medical tubing and an interior lumen therebetween.

18. An adaptor according to any of claims 14-17 , wherein the opening is a first opening and the at least two channels between the first strip and the film are in fluid communication with the first opening, further wherein the airway comprises a second opening at a first end of the second strip, and the at least two channels between the second strip and the film are in fluid communication with the second opening.

19. An adaptor according to claim 18, wherein the first opening is separated from the second opening by the film.

20. An adaptor according to any preceding claim, wherein the opening comprises a plurality of openings, each opening being in communication with the interior lumen.

21. An adaptor according to any preceding claim, wherein the sealing portion comprises a first wing and a second wing, wherein each wing extends outwardly from the first end of the airway.

22. An adaptor according to claim 21, wherein the upper sealing surface of each of the first wing and second wing extends outwardly from an uppermost surface of the airway, and the lower sealing surface of each of the first wing and second wing extends outwardly from a lowermost surface of the airway.

23. An adaptor according to claim 22, wherein the upper sealing surface of the first wing and lower sealing surface of the first wing converge toward each other.

24. An adaptor according to claim 22-23 , wherein the upper sealing surface of the second wing and lower sealing surface of the second wing converge toward each other.

25. An adaptor according to claim 22, wherein the lower sealing surface of the first wing and the lower sealing surface of the second wing are each planar.

26. A method of adapting a surgical wound dressing for use in negative pressure wound therapy, the surgical wound dressing having at least a peripheral adhesive skin contact layer on the underside of the surgical wound dressing, the method comprising:
a. providing an adaptor according to any preceding claim,
b. removing the release liner from the adhesive layer of the lower sealing surface of the airway; and
c. adhering the adaptor to an underside of a surgical wound dressing, such that the upper sealing surface of the sealing portion is adhered to the peripheral adhesive skin contact layer.

27. A method according to claim 26, the method further comprising adhering the peripheral adhesive skin contact layer of the surgical wound dressing to the periwound skin of a patient.

28. A method according to any of claims 26 or 27 , wherein the adhesive layer of the sealing portion and the adhesive periphery of the wound dressing together form a continuous adhesive boundary around a wound.

29. Use of an adaptor according to any of claims 1-25 in adapting a surgical wound dressing to a negative pressure wound dressing for use in negative pressure wound therapy.
